(19) United States
(12) Patent Application Publication
(10) Pub. No.: US 2026/0191976 A1
(43) Pub. Date: Jul. 9, 2026

(54) PROCESS FOR MAKING ANTIBODY-DRUG CONJUGATES

(71) Applicant: Merck Sharp & Dohme LLC, Rahway, NJ (US)

(72) Inventors: Patrick S. Fier, Monroe Township, NJ (US); Gregorio J. Estrada, Jersey City, NJ (US); Reed Larson, Bayonne, NJ (US); Tao Liang, Skillman, NJ (US); Patrick J. Moon, Rahway, NJ (US); Marc Poirier, Stewartsville, NJ (US); Gao Shang, Doylestown, PA (US); Michael Shevlin, Middlesex, NJ (US); Lu Wang, Scotch Plains, NJ (US)

(73) Assignee: Merck Sharp & Dohme LLC, Rahway, NJ (US)

(21) Appl. No.: 19/131,608

(22) PCT Filed: Dec. 6, 2023
(86) PCT No.: PCT/US2023/082742

§ 371 Date: May 21, 2025

Related U.S. Application Data
(60) Provisional application No. 63/430,898, filed on Dec. 7, 2022.

(51) Int. Cl. A61K 47/68; C07D 403/12; C07D 491/22

(52) U.S. Cl. CPC A61K 47/68037; A61K 47/6889; C07D 403/12; C07D 491/22

(57) ABSTRACT

The present disclosure is directed to methods for making compounds of structural formula (I): (I), wherein PG is defined above herein, which are useful for making antibody-drug conjugates. The present disclosure is also directed to processes for making related payload compounds, related Linker-Payload compounds, and to compounds that are useful as synthetic intermediates for making the compounds of formula (I).

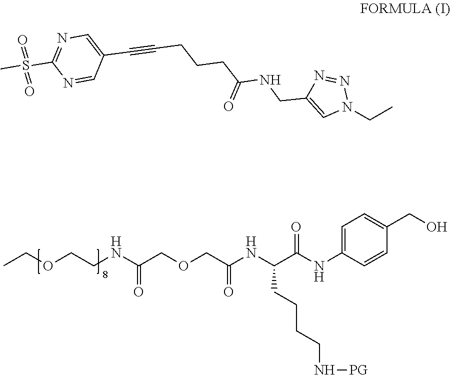

### CROSS-REFERENCE TO RELATED APPLICATIONS

This application claims the benefit of priority to U.S. Provisional Application No. 63/430,898, filed Dec. 7, 2022, the disclosure of which is incorporated herein by its entirety.

### FIELD

Provided herein are processes for making linker-payload moieties that are useful for making antibody-drug conjugates, wherein said antibody-drug conjugates are useful for the treatment of cancer. Also provided are processes for making related payload moieties, and compounds that are useful as synthetic intermediates for making the linker-payload moieties of the present disclosure.

### BACKGROUND

The global cancer burden has been continually rising and is predicted to keep growing throughout the coming decades, given population ageing and contemporary lifestyles. The American Cancer Society estimates that in 2022, 1.9 million new cancer cases will be diagnosed, and 609,360 cancer deaths will be recorded in the United States alone. The nearly 200 existing types of cancer share the fundamental hallmarks of uncontrolled growth and spread, due to the progressive acquirement of cellular capabilities of limitless proliferation and evasion from regulatory mechanisms.

Antibody-drug conjugates, or ADCs, are a class of highly potent biopharmaceutical drug composed of an antibody linked, via a chemical linker, to a biologically active drug or cytotoxic compound. These targeted agents combine the unique and very sensitive targeting capabilities of antibodies allowing sensitive discrimination between healthy and cancer tissues with the cell-killing ability of cytotoxic drugs.

Because these agents are capable of delivering highly cytotoxic payloads directly to tumor cells, they can be used to achieve high lethality toward the targeted cancer cells while minimizing harm to healthy cells.

Camptothecin (CPT) is a topoisomerase inhibitor, discovered in 1966 in systematic screening of natural products for anticancer drugs. It is isolated from the bark and stem of Camptotheca acuminata. CPT demonstrates anticancer activity in preliminary clinical trials, especially against breast, ovarian, colon, lung, and stomach cancers. However, it has low solubility, and adverse effects have been reported when used therapeutically, so synthetic and medicinal chemists have developed numerous syntheses of camptothecin, and various derivatives to increase the benefits of the chemical, with promising results. Four CPT analogues have been approved and are used in cancer chemotherapy today: topotecan, irinotecan, belotecan, and trastuzumab deruxtecan, which is an ADC that employs the camptothecin derivative deruxtecan as the payload. Numerous other camptothecin-based ADCs are currently being evaluated in human clinical trials, and further studies on this class of molecule are underway by several academic and industrial research groups, including clinical trials evaluating ADCs incorporating the linker-payload moiety depicted below (“Linker-Payload A”):

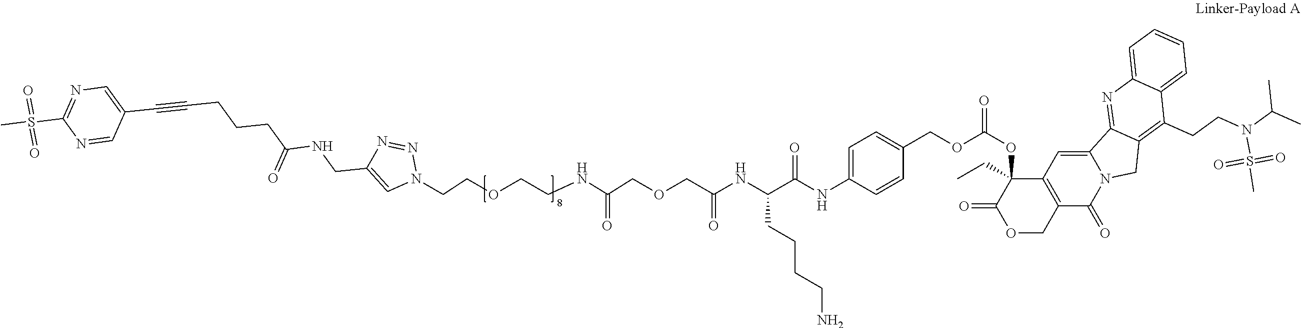

wherein the sulfone-substituted pyrimidinyl group serves as a conjugation handle, and which is disclosed in International Publication No. WO 2020/0347075

The success of targeting Trop-2 via antibody-drug conjugates (ADCs) in metastatic breast cancer (MBC), and urothelial cancer and ongoing trials in NSCLC have established Trop-2 targeting as a valid and fruitful strategy, and several Trop-2-targeted therapeutics have recently been developed for clinical use, such as anti-Trop-2 antibodies and Trop-2-targeted ADCs. Subsequently, multiple early-phase clinical trials have demonstrated good safety profiles, and clinical benefit associated with Trop-2-based ADCs across multiple tumor types. This includes clinical benefit, and tolerability in tumor types with limited treatment options, such as triple-negative breast cancer, platinum-resistant urothelial cancer, and small-cell lung cancer. An example of an ADC that is currently in early phase clinical trials is Immunoconjugate A, which incorporates Linker-Payload A:

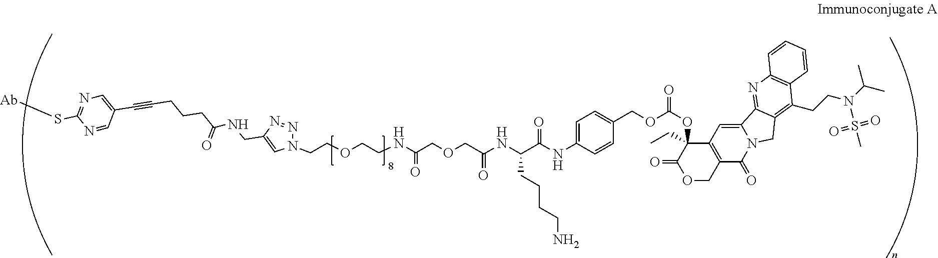

wherein Ab is an anti-Trop-2 antibody (sacituzumab), and n is an integer or decimal from 1 to 10, and which is described in US Patent Publication No. 20200347075.

There remains a need, however, for cost-effective synthetic routes for the preparation of such ADCs. This disclosure addresses that need.

### SUMMARY

The present disclosure is directed to a process for making the linker-payload moieties of formula (I), and for making ADCs incorporating these linker-payload moieties. The ADCs are useful for the treatment of cancer. In one aspect, the present disclosure provides a method (alternatively referred to herein as “Process A”) for preparing a compound of structural formula (I):

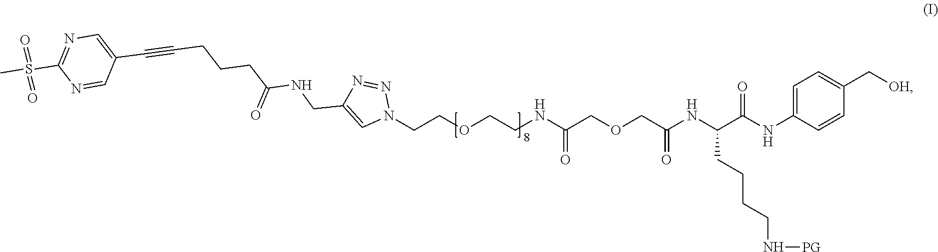

wherein PG is a primary amine protecting group,
comprising the steps:
   (A) contacting a compound of structural formula (i):

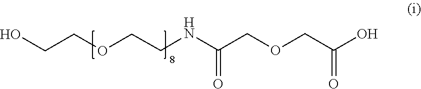

with a deoxychlorinating agent, in organic solvent A, at a temperature and for a time sufficient to form a compound of structural formula (ii):

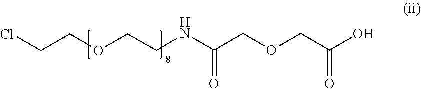

wherein organic solvent A is selected from dichloromethane, toluene, THF, acetonitrile, and mixtures thereof; and
   (B) contacting the product of Step A with a compound of structural formula (iii):

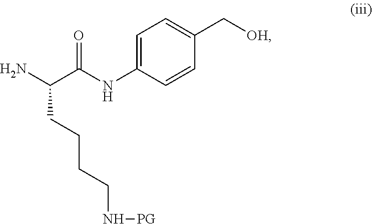

with an amide coupling agent, in the presence of a base, in organic solvent B, at a temperature, and for a time sufficient to form a compound of structural formula (iv):

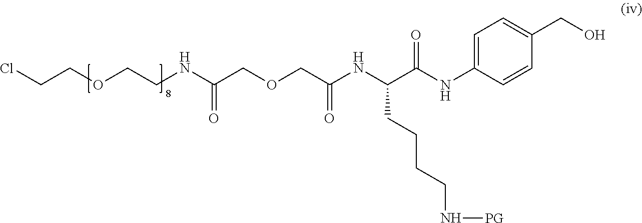

wherein organic solvent B is selected from methyl ethyl ketone, acetone, dichloroethane, dimethyl ether, diethyl ether, methyl isobutyl ketone, toluene, THF, DCM, MTBE, DMF, propylene carbonate, DME, 1,3-dimethyl-3,4,5,6-tetrahydro-2(1H)-pyrimidinone, 2-methyltetrahydrofuran, xylenes, ethyl acetate, NMP, anisole, isopropyl acetate, acetonitrile and mixtures thereof; and
   (C) contacting the product of step B with a compound of structural formula (v):

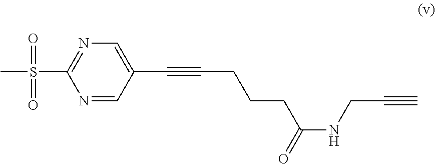 [SMILES: C#CCNC(=O)CCCC#Cc1cnc(S(C)(=O)=O)nc1]

in the presence of a copper salt, in the presence of an azide anion source, in organic solvent C, at a temperature and for a time sufficient to form the compound of structural formula (I), wherein organic solvent C is selected from THE, acetonitrile, toluene, methyl THF, isopropyl acetate, dichloromethane, water, and mixtures thereof.

In another aspect, the present disclosure provides novel synthetic intermediates useful in the processes of the present disclosure.

Other embodiments, aspects and features of the present disclosure are either further described in or will be apparent from the ensuing description, examples, and appended claims.

### DETAILED DESCRIPTION

The present disclosure is directed to processes for making the compound of structural formula (I), and for making ADCs using the compound of structural formula (I) as a linker. These ADCs are useful for treating cancer.

### Definitions and Abbreviations

In reference to the compounds employed as reactants or reagents in the processes of the present disclosure (e.g., Compounds (I), (II), (III), (i), (ii), (iii), etc.), a “stable” compound is one whose structure and properties remain or can be caused to remain essentially unchanged for a period of time sufficient to allow its use in the processes of the present disclosure so as to achieve the preparation of Compound of Formula (I), (II), (III), etc. In reference to Compounds of Formula (I), (II), (III) or Immunoconjugate A, a “stable” compound is a compound which can be prepared in accordance with the process of the present disclosure and then isolated and whose structure and properties remain or can be caused to remain essentially unchanged for a period of time sufficient to allow use of the compound for its intended purpose; e.g., for the therapeutic administration to a subject who has cancer, or in the case of the Compounds of Formula (I), to link a payload to an antibody as part of an antibody-drug conjugate

The term “salt(s)”, as employed herein, denotes acidic salts formed with inorganic and/or organic acids, as well as basic salts formed with inorganic and/or bases. In addition, when a depicted compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of a compound, starting material or synthetic intermediate of the present disclosure may be formed, for example, by reacting said compound, starting material or synthetic intermediate with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. 2^{nd} Ed. (2011) Zurich: Wiley-VCH; S. Berge et al., Journal of Pharmaceutical Sciences (1977) 66 (1) 1-19; P. Gould, International J. of Pharmaceutics (1986)33 201-217; Wermuth et al., The Practice of Medicinal Chemistry, 4^{th} Ed. (2015), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Stereochemically pure compounds may also be prepared by using chiral starting materials or by employing salt resolution techniques.

It is also possible that the compounds, starting materials and synthetic intermediates of the present disclosure may exist in different tautomeric forms, and all such forms are embraced within the scope of the present disclosure. For example, all keto-enol and imine-enamine forms of the compounds, starting materials and synthetic intermediates of the present disclosure are included.

All stereoisomers (for example, geometric isomers, optical isomers and the like) of the present compounds, starting materials and synthetic intermediates of the present disclosure (including those of the salts, solvates, hydrates and esters thereof), such as those which may exist due to asymmetric carbons on various substituents, including enantiomeric forms (which may exist even in the absence of asymmetric carbons), rotameric forms, atropisomers, and diastereomeric forms, are contemplated within the scope of the present disclosure. If a compound, starting material or synthetic intermediate of the present disclosure incorporates a double bond or a fused ring, both the cis- and trans-forms, as well as mixtures, are embraced within the scope of the present disclosure.

The following abbreviations are used below and have the following meanings: Ac is acetyl; anisole is methoxybenzene; Boc is tert-butoxycarbonyl; BOP is (benzotriazol-1-yloxytris(dimethylamino)phosphonium hexafluorophosphate); t-Bu is tertiary butyl; Cbz is benzyloxycarbonyl; Celite®545 is diatomaceous earth, flux-calcinated; COMU is (1-cyano-2-ethoxy-2-oxoethylidenaminooxy)dimethylamino-morpholino-carbenium hexafluorophosphate; CuBr—SMe₂ is copper (I) bromide dimethylsulfide complex; CUNO-5 is a carbon resin; DBU is 1,8-diazabicyclo[5.4.0]undec-7-ene; DCC is N,N′-dicyclohexylcarbodiimide; DCM is dichloromethane; DIEA or DIPEA is N,N-diisopropylethylamine; DMAc is dimethylacetamide; DMAP is dimethylaminopyridine; DMBCl is 2,2-Dimethylbut-3-ynoyl chloride; DME is 1,2-dimethoxyethane; DMF is N,N-dimethylformamide; DMSO is dimethyl sulfoxide; DPPA is diphenylphosphoryl azide; dppf is 1,1′-Bis(diphenylphosphino) ferrocene; EDC is 1-ethyl-3-(3-dimethylaminopropyl) carbodiimide; EEDQ is 2-Ethoxy-1-ethoxycarbonyl-1,2-dihydroquinoline; EtOAc is ethyl acetate; Fmoc is fluorenylmethoxycarbonyl; HATU is (1-[bis(dimethylamino)methylene]-1H-1,2,3-triazolo[4,5-b]pyridinium 3-oxide hexafluorophosphate; HBTU is (2-(1H-benzotriazol-1-yl)-1,1,3,3-tetramethyluronium hexafluorophosphate; HPLC is high performance liquid chromatography; IPAc is isopropyl acetate; 2,6-lutidine is 2,6-dimethylpyridine; LC/MS is liquid chromatography/Mass Spectrometry; Me is methyl; MeCN is acetonitrile; Me-THF (and methyl THF) are 2-methyltetrahydrofuran; mesyl is methanesulfonyl; MMT is monomethoxytrityl; MTBE is tert-butyl methyl ether; NaMSA is methanesulfonic acid sodium salt; NMP is N-methyl-2-pyrrolidone; [Pd₂(dba)₃] is Tris(dibenzylideneacetone)dipalladium(0); 2-picoline is 2-methylpyridine; TBAF is tetrabutylammonium fluoride; TEA is triethylamine; THF is tetrahydrofuran; tosyl is p-toluenesulfonyl; and TSTU is O—(N-succinimidyl)-N,N,N′,N′-tetramethyluronium tetrafluoroborate.

### The Processes of the Present Disclosure

The present disclosure is directed to processes for making compounds of structural formula (I), useful as a linker in ADC molecules. One aspect of the present disclosure is the following process (“Process A”) for making compounds of structural formula (I):

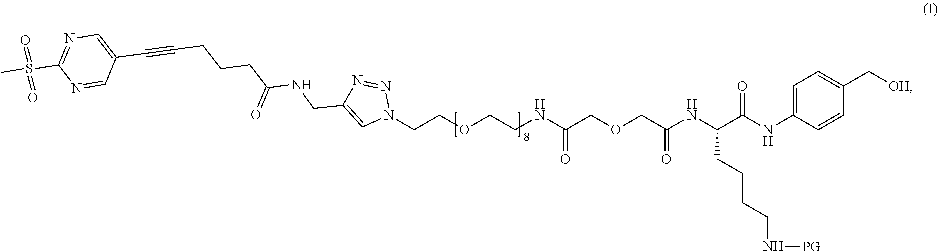

wherein PG is a primary amine protecting group, and said process comprises the steps:
   (A) contacting a compound of structural formula (i):

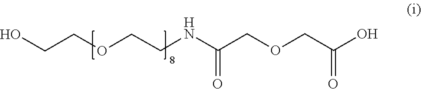

with a deoxychlorinating agent, in organic solvent A, at a temperature and for a time sufficient to form a compound of structural formula (ii):

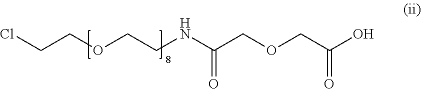

wherein organic solvent A is selected from dichloromethane, toluene, THF, acetonitrile, and mixtures thereof; and
   (B) contacting the product of Step A with a compound of structural formula

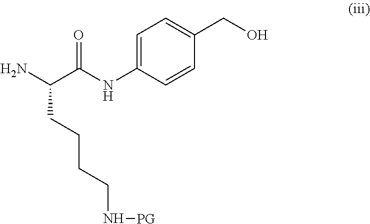

with an amide coupling agent, in the presence of a base, in organic solvent B, at a temperature, and for a time sufficient to form a compound of structural formula (iv):

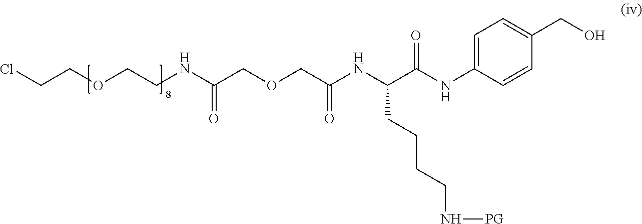

and wherein organic solvent B is selected from methyl ethyl ketone, acetone, dichloroethane, dimethyl ether, diethyl ether, methyl isobutyl ketone, toluene, THF, DCM, MTBE, DMF, propylene carbonate, DME, 1,3-dimethyl-3,4,5,6-tetrahydro-2(1H)-pyrimidinone, 2-methyltetrahydrofuran, xylenes, ethyl acetate, NMP, anisole, isopropyl acetate, acetonitrile and mixtures thereof; and
   (C) contacting the product of step B with a compound of structural formula (v):

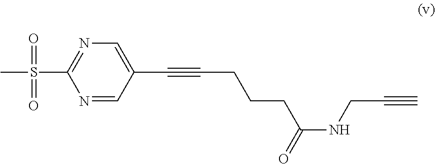 [SMILES: C#CCNC(=O)CCCC#Cc1cnc(S(C)(=O)=O)nc1]

in the presence of a copper salt, and an azide anion source, in organic solvent C, at a temperature and for a time sufficient to form a compound of structural formula (I), wherein organic solvent C is selected from THE, acetonitrile, toluene, methyl THF, isopropyl acetate, dichloromethane, water, and mixtures thereof.

In one embodiment, for Process A, the deoxychlorinating agent used in Step (A) is selected from SOCl₂, PCl₃, PCl₅, POCl₃, oxalyl chloride, tosyl chloride, mesyl chloride, triphenylphosphine/CCl₄, and triphenyl phosphine/hexachloroethane.

In another embodiment, for Process A, the primary amine protecting group is selected from Boc, MMT, Cbz, and Fmoc.

In another embodiment, for Process A, the deoxychlorinating agent used in Step (A) is SOCl₂ or oxalyl chloride.

In one embodiment, for Process A, organic solvent A is dichloromethane or THF.

In one embodiment, for Process A, the product of Step (A) is isolated prior to performing Step (B).

In another embodiment, for Process A, the product of Step (A) is isolated, purified, and dried prior to performing Step (B).

In one embodiment, for Process A, the amide coupling agent used in Step (B) is selected from HATU, EDC, DCC, HBTU, BOP, COMU, and TSTU.

In another embodiment, for Process A, the amide coupling agent used in Step (B) is selected from HATU, EDC, and DCC.

In one embodiment, for Process A, the base used in Step (B) is selected from a carbonate base, TEA, DIPEA, DBU, pyridine, and 2-picoline.

In another embodiment, for Process A, the base used in Step (B) is K₂CO₃, or Na₂CO₃.

In one embodiment, for Process A, the primary amine protecting group in Step (B) is selected from Boc, MMT, Cbz, or Fmoc.

In one embodiment, for Process A, the organic solvent B is selected from methyl ethyl ketone, acetone, diethyl ether, THF, DCM, and NMP.

In one embodiment, for Process A, the product of Step (B) is isolated prior to performing Step (C).

In another embodiment, for Process A, the product of Step (B) is isolated and purified prior to performing Step (C).

In one embodiment, for Process A, the copper salt used in Step (C) is selected from CuCl, CuBr, CuBr—SMe₂, CuI, Cu(OAc), Cu(OAc)₂, CuSO₄, Cu₃(PO₄)₂.

In one embodiment, for Process A, the azide anion source in Step (C) is NaN₃ or KN₃.

In one embodiment, for Process A, organic solvent C is dichloromethane, water, THF, or a mixture thereof.

In one embodiment, for Process A, the product of Step (C) is isolated.

In another embodiment, for Process A, the product of Step (C) is isolated and purified.

In one embodiment, for Process A, the product of Step C can be adsorbed onto a solid phase support for ease of use in subsequent chemical reactions. In another embodiment, the solid phase support is a high surface area microporous solid. In a specific embodiment, the solid phase support is Celite or cellulose. In a preferred embodiment, the solid phase support is Celite.

In another aspect, the present disclosure provides an alternate process (“Process B”) for making a compound of formula (I):

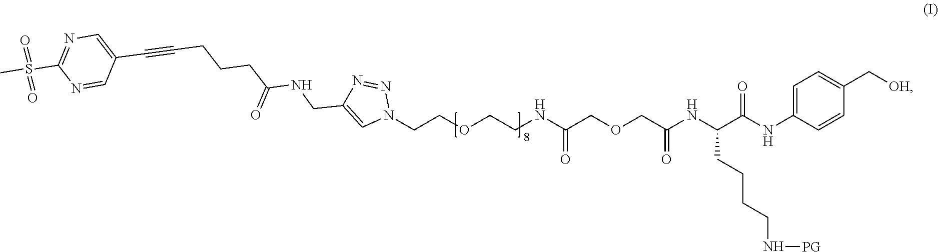

wherein PG is a primary amine protecting group,
comprising the steps:
   (A) contacting a compound of structural formula (vi):

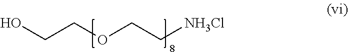

with a deoxychlorinating agent, in organic solvent D, at a temperature and for a time sufficient to form a compound of structural formula (vii):

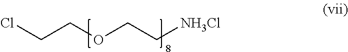

wherein organic solvent D is selected from dichloromethane, toluene, THF, acetonitrile, and mixtures thereof;
   (B) contacting the product of Step A with diglycolic anhydride (viii):

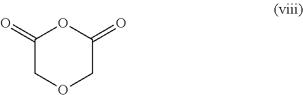

in organic solvent E, at a temperature, and for a time sufficient to form a compound of structural formula (ix):

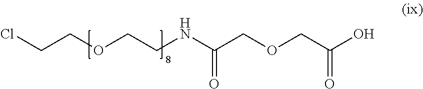

wherein organic solvent E is selected from THE, acetonitrile, toluene, methyl THF, isopropyl acetate, ethyl acetate, dichloromethane, and mixtures thereof;
   (C) contacting the product of Step B with a compound of structural formula (iii):

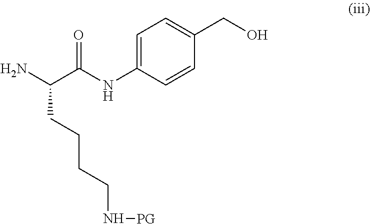

with an amide coupling agent, in the presence of a base, in organic solvent F, at a temperature, and for a time sufficient to form a compound of structural formula (iv):

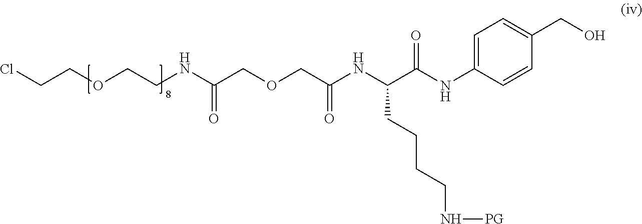

wherein organic solvent F is selected from methyl ethyl ketone, acetone, dichloroethane, dimethyl ether, diethyl ether, methyl isobutyl ketone, toluene, THF, DCM, MTBE, DMF, propylene carbonate, DME, 1,3-dimethyl-3,4,5,6-tetrahydro-2(1H)-pyrimidinone, 2-methyltetrahydrofuran, xylenes, ethyl acetate, NMP, anisole, isopropyl acetate, acetonitrile and mixtures thereof; and
   (D) contacting the product of Step C with an azide anion, in organic solvent G, at a temperature and for a time sufficient to form an intermediate compound of structural formula (x):

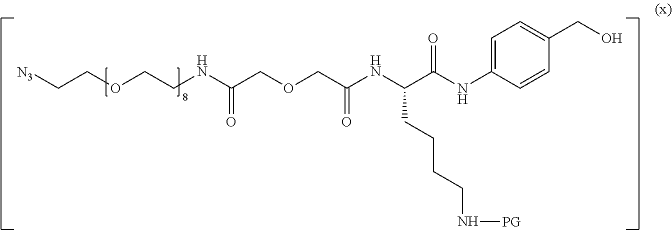

then contacting intermediate compound (x) with a compound of structural formula (v):

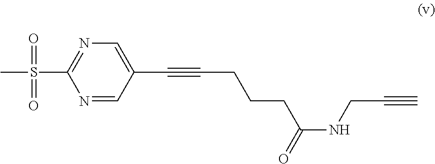 [SMILES: C#CCNC(=O)CCCC#Cc1cnc(S(C)(=O)=O)nc1]

in the presence of a copper salt, at a temperature and for a time sufficient to form the compound of structural formula (I), wherein organic solvent G is selected from THE, acetonitrile, toluene, methyl THF, isopropyl acetate, ethyl acetate, dichloromethane, water, and mixtures thereof.

In one embodiment, for Process B, the deoxychlorinating agent used in Step (A) is selected from SOCl₂, PCl₃, PCl₅, POCl₃, oxalyl chloride, tosyl chloride, mesyl chloride, triphenylphosphine/CCl₄, and triphenyl phosphine/hexachloroethane.

In another embodiment, for Process B, the deoxychlorinating agent used in Step (A) is SOCl₂ or oxalyl chloride.

In another embodiment, for Process B, the primary amine protecting group is selected from Boc, MMT, Cbz, and Fmoc.

In one embodiment, for Process B, organic solvent D is dichloromethane or THF.

In one embodiment, for Process B, the product of Step (A) is isolated prior to performing Step (B).

In another embodiment, for Process B, the product of Step (A) is isolated and purified prior to performing Step (B).

In one embodiment, for Process B, organic solvent E is selected from methyl ethyl ketone, acetone, diethyl ether, THF, DCM, and NMP.

In one embodiment, for Process B, the product of Step (B) is isolated prior to performing Step (C).

In another embodiment, for Process B, the product of Step (B) is isolated and purified prior to performing Step (C).

In one embodiment, for Process B, the amide coupling agent used in Step (C) is selected from HATU, EDC, DCC, HBTU, BOP, COMU, and TSTU.

In another embodiment, for Process B, the amide coupling agent used in Step (C) is selected from HATU, EDC, and DCC.

In one embodiment, for Process B, the base used in Step (C) is selected from a carbonate base, TEA, DIPEA, DBU, pyridine, and 2-picoline.

In another embodiment, for Process B, the base used in Step (C) is K₂CO₃, or Na₂CO₃.

In one embodiment, for Process B, organic solvent F is selected from methyl ethyl ketone, acetone, diethyl ether, THF, DCM, and NMP.

In one embodiment, for Process B, the product of Step (C) is isolated prior to performing Step (D).

In another embodiment, for Process B, the product of Step (C) is isolated and purified prior to performing Step (D).

In one embodiment, for Process B, the source of the azide anion in Step (D) is NaN₃ or KN₃.

In another embodiment, for Process B, the copper salt used in Step (D) is selected from CuCl, CuBr, CuBr—SMe₂, CuI, Cu(OAc), Cu(OAc)₂, CuSO₄, Cu₃(PO₄)₂.

In one embodiment, for Process B, organic solvent G is dichloromethane, water, THF, or a mixture thereof.

In one embodiment, for Process B, intermediate compound (x) is not isolated during Step (D).

In one embodiment, for Process B, the product of Step D is isolated.

In another embodiment, for Process B, the product of Step D is isolated and purified.

In one embodiment, for Process B, the product of Step D can be adsorbed onto a solid phase support for ease of use in subsequent chemical reactions. In another embodiment, the solid phase support is a high surface area microporous solid. In a specific embodiment, the solid phase support is Celite or cellulose. In a preferred embodiment, the solid phase support is Celite.

In another aspect, the present disclosure provides another process (“Process C”) for making a compound of formula (I):

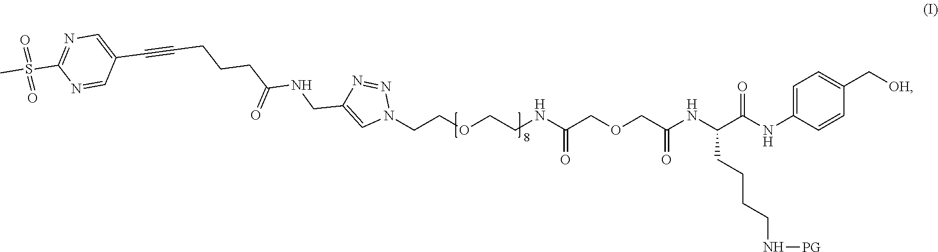

wherein PG is a primary amine protecting group,
comprising the steps:
   (A) contacting a compound of structural formula (vii):

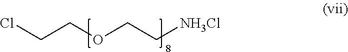

with an azide anion, in organic solvent H, at a temperature and for a time sufficient to form an intermediate compound of structural formula (xii):

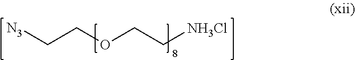

then, contacting the intermediate compound (xii) with a compound of structural formula (v):

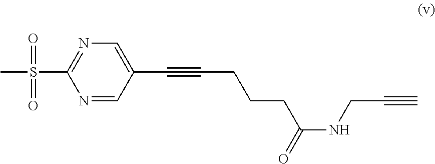 [SMILES: C#CCNC(=O)CCCC#Cc1cnc(S(C)(=O)=O)nc1]

in the presence of a copper salt, at a temperature and for a time sufficient to form compound of structural formula (xiii):

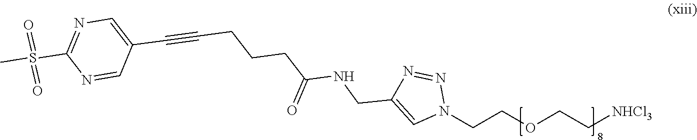

wherein organic solvent H is selected from THF, acetonitrile, DMF, toluene, methyl THF, isopropyl acetate, ethyl acetate, dichloromethane, water, and mixtures thereof, and
   (B) contacting the product of Step A with a compound of structural formula (xiv):

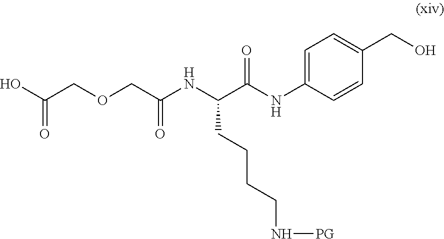

in the presence of an amide coupling agent and a base in organic solvent I at a temperature, and for a time sufficient to form the compound of structural formula (I), wherein the organic solvent I is selected from THE, acetonitrile, toluene, methyl THF, isopropyl acetate, ethyl acetate, dichloromethane, and mixtures thereof.

In one embodiment, for Process C, the primary amine protecting group is selected from Boc, MMT, Cbz, and Fmoc.

In one embodiment, for Process C, the source of the azide anion in Step (A) is NaN₃ or KN₃.

In one embodiment, for Process C, the copper salt used in Step (A) is selected from CuCl, CuBr, CuBr—SMe₂, CuI, Cu(OAc), Cu(OAc)₂, CuSO₄, Cu₃(PO₄)₂.

In one embodiment, for Process C, organic solvent H is a selected from dichloromethane, DMF, THF, or mixtures thereof.

In one embodiment, for Process C, intermediate (xii) is not isolated during Step A.

In one embodiment, for Process C, organic solvent H is a selected from dichloromethane, DMF, THF, and mixtures thereof.

In one embodiment, for Process C, the product of Step (A) is isolated prior to performing Step (B).

In another embodiment, for Process C, the product of Step (A) is isolated and purified prior to performing Step (B).

In one embodiment, for Process C, the amide coupling agent used in Step (B) is selected from HATU, EDC, DCC, HBTU, BOP, COMU, and TSTU.

In another embodiment, for Process C, the amide coupling agent used in Step (B) is selected from HATU, EDC, and DCC.

In one embodiment, for Process C, the base used in Step (B) is selected from a carbonate base, TEA, DIPEA, DBU, pyridine, and 2-picoline.

In another embodiment, for Process C, the base used in Step (B) is DBU, K₂CO₃, or Na₂CO₃.

In one embodiment, for Process C, organic solvent I is selected THF, acetonitrile, and dichloromethane.

In one embodiment, for Process C, the product of Step (B) is isolated.

In another embodiment, for Process C, the product of Step (B) is isolated and purified.

In one embodiment, for Process C, the product of Step B can be adsorbed onto a solid phase support for ease of use in subsequent chemical reactions. In another embodiment, the solid phase support is a high surface area microporous solid. In a specific embodiment, the solid phase support is Celite or cellulose. In a preferred embodiment, the solid phase support is Celite.

In another aspect, the present disclosure provides another alternate process (“Process D”), for making a compound of formula (I):

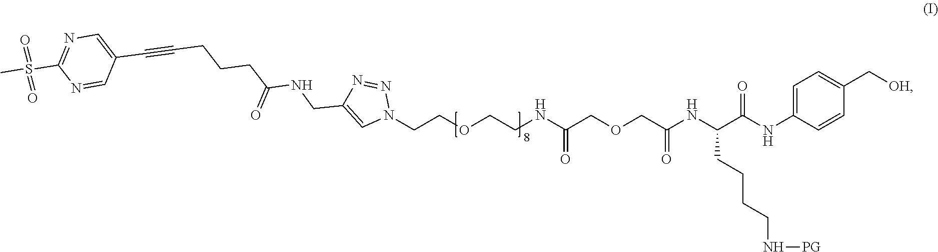

wherein PG is a primary amine protecting group,
comprising the steps:
   (A) contacting a compound of structural formula (ii):

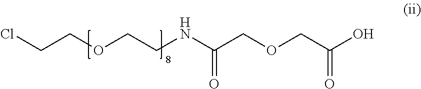

with an azide anion, in organic solvent J, at a temperature and for a time sufficient to form an intermediate compound of structural formula (xv):

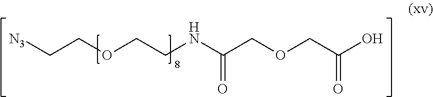

then, contacting the intermediate compound (xv) with a compound of structural formula (v):

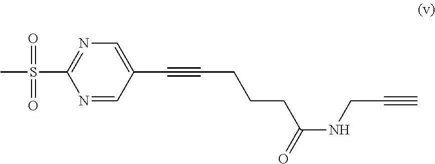 [SMILES: C#CCNC(=O)CCCC#Cc1cnc(S(C)(=O)=O)nc1]

in the presence of a copper salt, at a temperature and for a time sufficient to form a compound of structural formula (xvi):

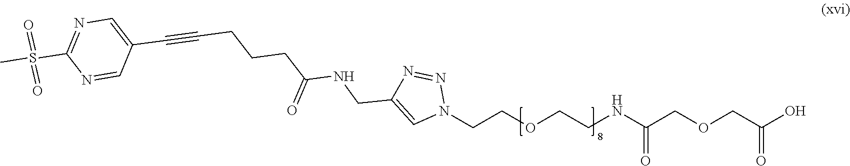

wherein organic solvent J is selected from THE, acetonitrile, toluene, methyl THF, isopropyl acetate, ethyl acetate, dichloromethane, water, and mixtures thereof, and
   (B) contacting the product of Step A with a compound of structural formula (iii):

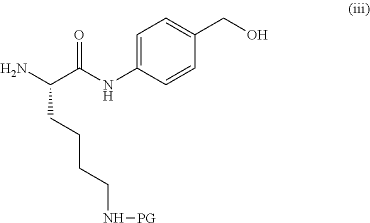

in the presence of an amide coupling agent and a base, in organic solvent K, at a temperature, and for a time sufficient to form a compound of structural formula (I), wherein organic solvent K is selected from THE, acetonitrile, toluene, methyl THF, isopropyl acetate, ethyl acetate, dichloromethane, and mixtures thereof.

In one embodiment, for Process D, the primary amine protecting group is selected from Boc, MMT, Cbz, and Fmoc.

In one embodiment, for Process D, the source of the azide anion in Step (A) is NaN₃ or KN₃.

In one embodiment, for Process D, the copper salt used in Step (A) is selected from CuCl, CuBr, CuBr—SMe₂, CuI, Cu(OAc), Cu(OAc)₂, CuSO₄, Cu₃(PO₄)₂.

In one embodiment, for Process D, organic solvent J is a selected from dichloromethane, THF, and acetonitrile.

In one embodiment, for Process D, Step (A), intermediate (xv) is not isolated.

In one embodiment, for Process D, the product of Step (A) is isolated prior to performing Step (B).

In another embodiment, for Process D, the product of Step (A) is isolated and purified prior to performing Step (B).

In one embodiment, for Process D, the amide coupling agent used in Step (B) is selected from HATU, EDC, DCC, HBTU, BOP, COMU, and TSTU.

In another embodiment, for Process D, the amide coupling agent used in Step (B) is selected from HATU, EDC, and DCC.

In one embodiment, for Process D, the base used in Step (B) is selected from a carbonate base, TEA, DIPEA, DBU, pyridine, and 2-picoline.

In another embodiment, for Process D, the base used in Step (B) is DBU, K₂CO₃, or Na₂CO₃.

In one embodiment, for Process D, organic solvent K is selected from THE, DCM, and acetonitrile.

In one embodiment, for Process D, the product of Step (B) is isolated.

In another embodiment, for Process D, the product of Step (B) is isolated and purified.

In one embodiment, for Process D, the product of Step B can be adsorbed onto a solid phase support for ease of use in subsequent chemical reactions. In another embodiment, the solid phase support is a high surface area microporous solid. In a specific embodiment, the solid phase support is Celite or cellulose. In a preferred embodiment, the solid phase support is Celite.

In another aspect, the present disclosure provides another alternate process (“Process E”), for making a compound of formula (I):

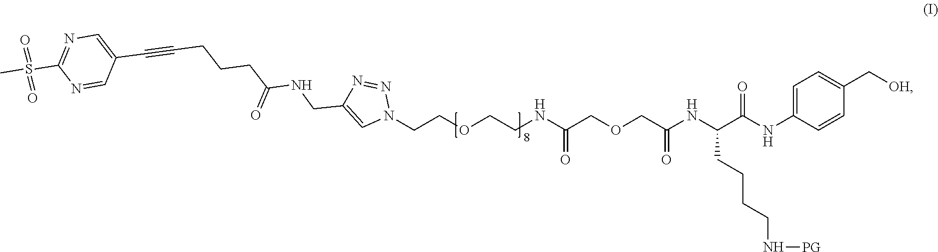

wherein PG is a primary amine protecting group,
comprising the steps:
   (A) contacting a compound of structural formula (xv):

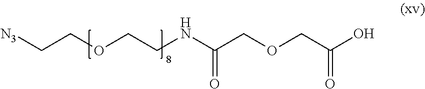

with a compound of structural formula (iii):

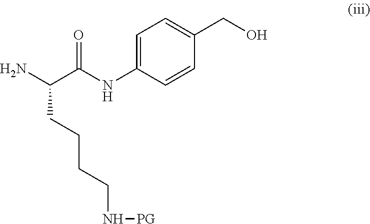

wherein PG is a primary amine protecting group, with an amide coupling agent, in organic solvent L, at a temperature, and for a time sufficient to form a compound of structural formula (x):

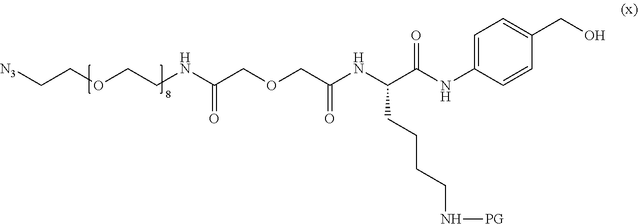

wherein PG is a primary amine protecting group, and organic solvent L is selected from methyl ethyl ketone, acetone, dichloroethane, dimethyl ether, diethyl ether, methyl isobutyl ketone, toluene, THF, DCM, MTBE, DMF, propylene carbonate, DME, 1,3-dimethyl-3,4,5,6-tetrahydro-2 (1H)-pyrimidinone, 2-methyltetrahydrofuran, xylenes, ethyl acetate, NMP, anisole, isopropyl acetate, acetonitrile and mixtures thereof; and
   (B) contacting the product of step A with a compound of structural formula (v):

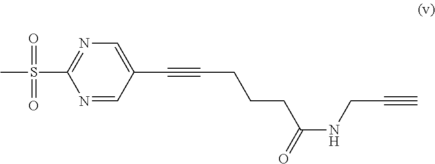 [SMILES: C#CCNC(=O)CCCC#Cc1cnc(S(C)(=O)=O)nc1]

in the presence of a copper salt, in organic solvent M, at a temperature and for a time sufficient to form the compound of structural formula (I), wherein organic solvent M is selected from methyl ethyl ketone, acetone, dichloroethane, dimethyl ether, diethyl ether, methyl isobutyl ketone, toluene, THF, DCM, MTBE, DMF, propylene carbonate, DME, 1,3-dimethyl-3,4,5,6-tetrahydro-2(1H)-pyrimidinone, 2-methyltetrahydrofuran, xylenes, ethyl acetate, NMP, anisole, isopropyl acetate, acetonitrile and mixtures thereof.

In one embodiment, for Process E, the primary amine protecting group is selected from Boc, MMT, Cbz, and Fmoc.

In one embodiment, for Process E, organic solvent L in Step (A) is THF.

In one embodiment, for Process E, the product of Step (A) is not isolated prior to performing Step (B).

In one embodiment, for Process E, the product of Step (A) is prepared in solution and used in solution in Step (B).

In one embodiment, for Process E, the amide coupling agent used in Step (A) is selected from EEDQ, HATU, EDC, DCC, HBTU, BOP, COMU, and TSTU.

In another embodiment, for Process E, the amide coupling agent used in Step (A) is EEDQ.

In one embodiment, for Process E, the primary amine protecting group is selected from Boc, MMT, Cbz, and Fmoc.

In another embodiment, for Process E, the primary amine protecting group is Boc.

In one embodiment, for Process E, Step (B), organic solvent M is THF.

In one embodiment, for Process E, the copper salt used in Step (B) is selected from CuCl, CuBr, CuBr—SMe₂, CuI, Cu(OAc), Cu(OAc)₂, CuSO₄, Cu₃(PO₄)₂.

In another embodiment, for Process E, the copper salt used in Step (B) is CuBr—SMe₂.

In one embodiment, for Process E, the product of Step (B) is isolated.

In one embodiment, for Process E, the product of Step (B) is isolated and purified.

In one embodiment, the product of Step B can be adsorbed onto a solid phase support for ease of use in subsequent chemical reactions. In another embodiment, the solid phase support is a high surface area microporous solid. In a specific embodiment, the solid phase support is Celite or cellulose. In a preferred embodiment, the solid phase support is Celite.

In another aspect, the present disclosure provides a process (“Process F”) for making a linker-payload moiety of structural formula (II):

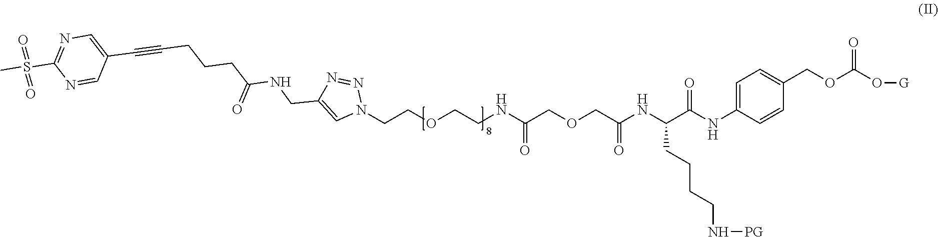

wherein G is a cytotoxic drug payload, PG is a primary amine protecting group comprising the steps:
   (A) contacting a drug that contains an —OH group, represented by (xvii):

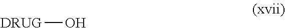

with triphosgene, in the presence of a base, in organic solvent N, at a temperature and for a time sufficient to form the compound of structural formula (xviii):

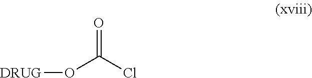

wherein organic solvent N is selected from methyl ethyl ketone, acetone, dichloroethane, dimethyl ether, diethyl ether, methyl isobutyl ketone, toluene, THF, DCM, MTBE, propylene carbonate, DME, 1,3-dimethyl-3,4,5,6-tetrahydro-2(1H)-pyrimidinone, 2-methyltetrahydrofuran, xylenes, ethyl acetate, NMP, anisole, isopropyl acetate, acetonitrile and mixtures thereof; and
   (B) contacting the product of Step A with a compound of formula (I):

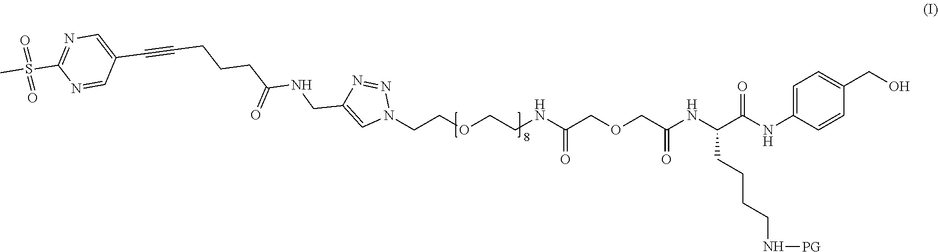

in the presence of a base, in organic solvent O, at a temperature and for a time sufficient to form the compound having the formula (II), wherein organic solvent O is selected from methyl ethyl ketone, acetone, dichloroethane, dimethyl ether, diethyl ether, methyl isobutyl ketone, toluene, THF, DCM, MTBE, DMF, propylene carbonate, DME, 1,3-dimethyl-3,4,5,6-tetrahydro-2(1H)-pyrimidinone, 2-methyltetrahydrofuran, xylenes, ethyl acetate, NMP, anisole, isopropyl acetate, acetonitrile and mixtures thereof.

In one embodiment, for Process F, the base used in Step (A) is selected from DMAP, TEA, pyridine, and 2,6-lutidine.

In one embodiment, for Process F, is a selected from dichloromethane, DMF, THF, and mixtures thereof.

In one embodiment, for Process F, the product of Step (A) is not isolated prior to performing Step (B).

In another embodiment, for Process F, the product of Step (A) is isolated prior to performing Step (B).

In another embodiment, for Process F, the product of Step (A) is isolated and purified prior to performing Step (B).

In another embodiment, for Process F, the reaction mixture of Step (A) is directly used in Step (B).

In one embodiment, for Process F, the base used in Step (B) is selected from DMAP, TEA, pyridine, and 2,6-lutidine.

In one embodiment, for Process F, organic solvent O is organic solvent M is selected from dichloromethane, DMF, THF, and mixtures thereof.

In another embodiment, organic solvent N is the same as organic solvent O.

In one embodiment, for Process F, the product of Step (B) is isolated.

In another embodiment, for Process F, the product of Step (B) is isolated and purified.

In one embodiment, for Process F, the product of Step B can be adsorbed onto a solid phase support for ease of use in subsequent chemical reactions. In another embodiment, the solid phase support is a high surface area microporous solid. In a specific embodiment, the solid phase support is Celite or cellulose. In a preferred embodiment, the solid phase support is Celite.

In one embodiment, for Process F, in Step A, compound xvii has structure (xix):

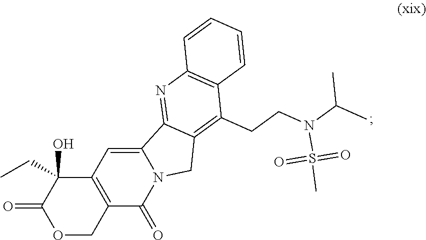 [SMILES: CC[C@@]1(O)C(=O)OCc2c1cc1n(c2=O)Cc2c-1nc1ccccc1c2CCN(C(C)C)S(C)(=O)=O]

in Step B, compound xviii, has the structure (xx):

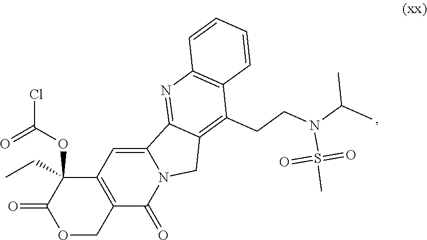 [SMILES: CC[C@@]1(OC(=O)Cl)C(=O)OCc2c1cc1n(c2=O)Cc2c-1nc1ccccc1c2CCN(C(C)C)S(C)(=O)=O]

and the product of Step B is Compound (xxi):

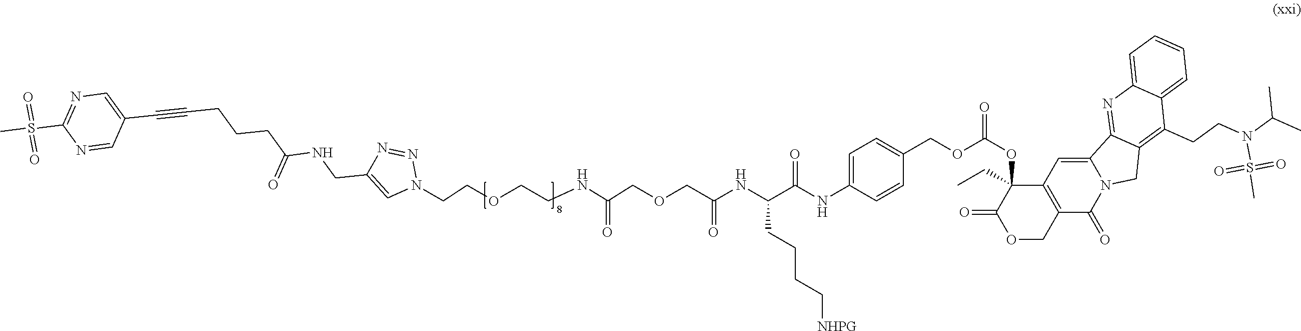

In one embodiment, the compound of formula (xxi) is deprotected to provide Linker-Payload A:

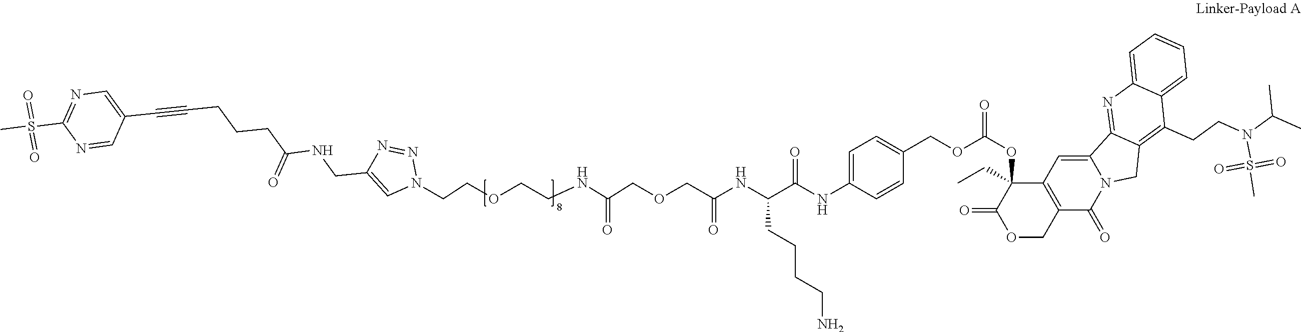

The compounds of formula (II) may be deprotected using well-known methods, then converted to the corresponding antibody-drug conjugate by conjugation to an antibody, using well-known conjugation methods, including those disclosed in International Publication Nos. WO 2018/025168, WO 2019/11466, and WO 2020/0347075.

In one embodiment, a compound of formula (II) can be deprotected to provide a compound of formula (xxii):

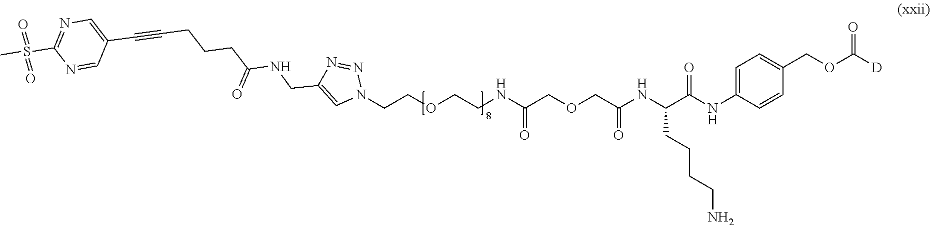

wherein G is a drug, then subsequently conjugated with an antibody or antibody fragment, to make an antibody-drug conjugate of formula (III):

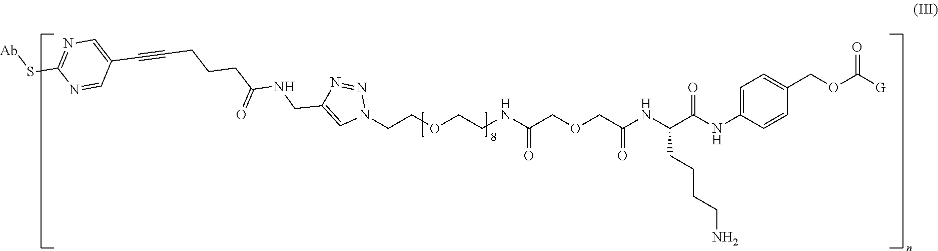

wherein n is a decimal from 0 to 8, Ab is an antibody or antibody fragment, G is a cytotoxic drug payload, and the sulfur atom in Formula (III) is from a thiol group on the antibody.

In one embodiment, a Compound of formula (xxi) can be deprotected, then conjugated with a monoclonal antibody, to make an antibody-drug conjugate of formula (xxiii):

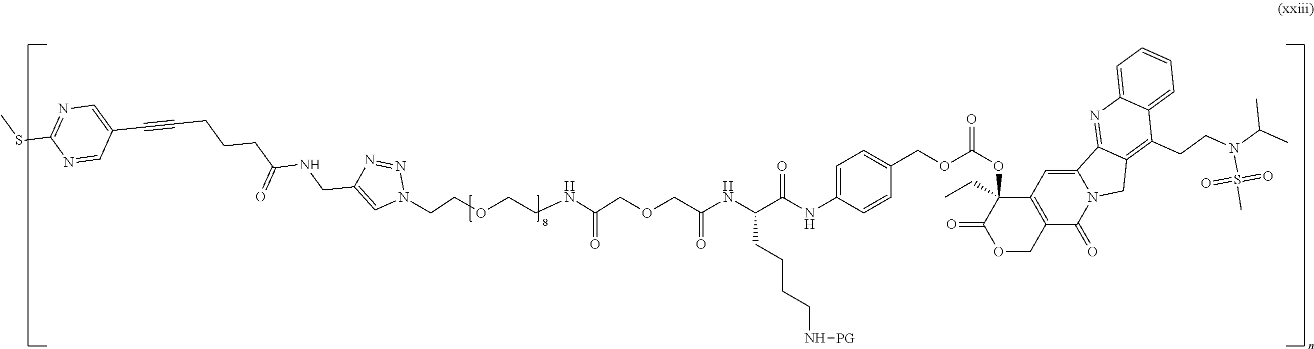

wherein PG is primary amine protecting group, n is an integer from 1 to 8, Ab is an antibody or antibody fragment, and the sulfur atom in Formula (xxiii) is from a thiol group on the antibody.

Compound (xix) can be made using methods disclosed in International Publication No. WO 2020/0347075, or using the methods described herein.

In one embodiment, for the antibody-drug conjugate of formula (xxiii)

In another aspect, the present disclosure provides a process (“Process G”) for making a payload compound of formula (xix):

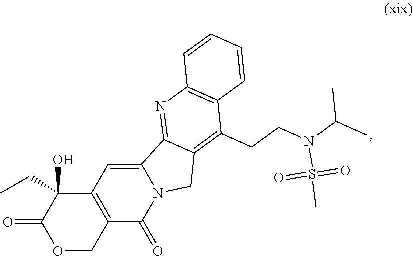 [SMILES: CC[C@@]1(O)C(=O)OCc2c1cc1n(c2=O)Cc2c-1nc1ccccc1c2CCN(C(C)C)S(C)(=O)=O]

comprising contacting a compound of structural formula (xxiv) (camptothecin):

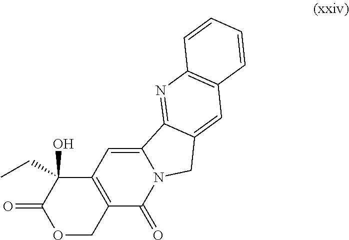 [SMILES: CC[C@@]1(O)C(=O)OCc2c1cc1n(c2=O)Cc2cc3ccccc3nc2-1]

with a compound of structural formula (xxv):

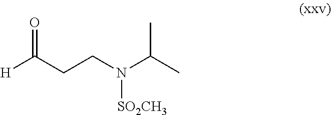

in the presence of an acid, a metal salt, and an oxidizing agent, in solvent P, at a temperature, and for a time sufficient to form the payload compound of structural formula xix, wherein solvent P is selected from water, MeCN, acetic acid, trifluoroethanol, NMP, propylene glycol, trifluoroacetic acid, and mixtures thereof.

In one embodiment for Process G, the acid used is selected from H₂SO₄, TFA, phosphoric acid, sulfonic acid, and methanesulfonic acid.

In one embodiment for Process G, the metal salt used in Step (A) is selected from Fe₂SO₄, V(acac)₃, V₂O₃, NaVO₃, FeCl₃, and FeO.

In one embodiment, for Process G, the oxidizing agent used is selected from H₂O₂, a persulfate, peracetic acid, cumene hydrogen peroxide, 2-butanone peroxide, oxone, urea hydrogen peroxide, sodium perborate, di-t-butyl peroxide, dibenzoyl peroxide, and sodium hypochlorite.

In one embodiment for Process G, solvent P is water.

In one embodiment for Process G, the product is isolated.

In another embodiment for Process G, the product is isolated and purified.

### Synthetic Intermediates

The present disclosure also provides compounds that are useful as synthetic intermediates in the claimed processes for making the compounds of the present disclosure.

Examples of such synthetic intermediates include, but are not limited to, the following compounds:

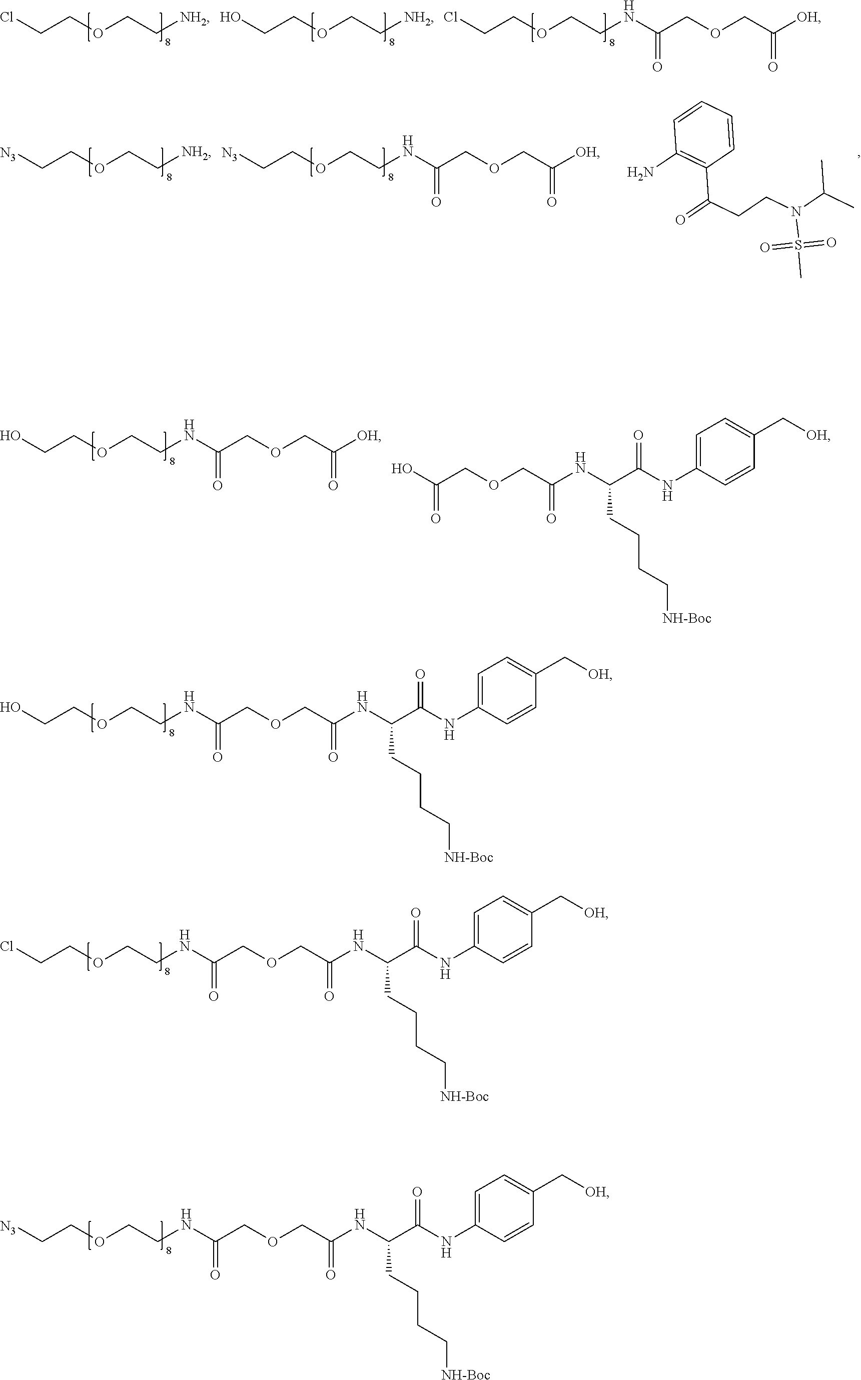

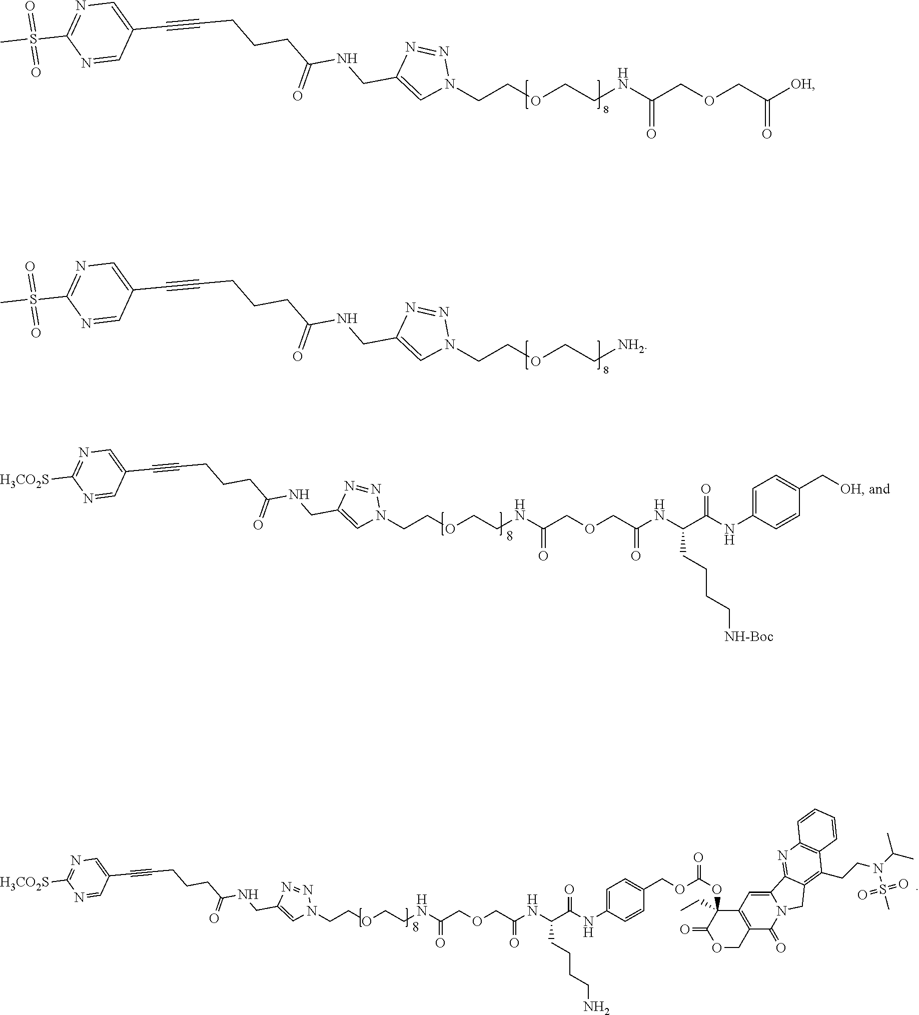

### Methods for Practicing the Processes of the Present Disclosure

The processes described herein, including starting materials, and synthetic intermediates, may be carried out using known or readily prepared starting materials, following methods known to one skilled in the art of organic synthesis. Methods useful for practicing the processes of the present disclosure are set forth herein. Alternative synthetic pathways and analogous structures will be apparent to those skilled in the art of organic synthesis.

One skilled in the art of organic synthesis will recognize that the processes of the present disclosure may require protection of certain functional groups (i.e., derivatization for the purpose of chemical compatibility with a particular reaction condition). Suitable protecting groups for the various functional groups of starting materials, intermediates, and products of the processes of the present disclosure, and methods useful for the installation and removal of these protecting groups are well known in the art of organic chemistry. A summary of many of these methods can be found in Wuts et al., Protective Groups in Organic Synthesis, Wiley-Interscience, New York, 5th Ed. (2014).

One skilled in the art of organic synthesis will also recognize that one of the processes disclosed herein may be more desirable depending on the choice of appendage substituents.

Additionally, one skilled in the art will recognize that in some cases the order of reactions of the processes of the present disclosure may differ from that presented herein to avoid functional group incompatibilities and thus adjust the synthetic route accordingly.

The starting materials and synthetic intermediates prepared using the processes set forth herein may be isolated and purified if desired using conventional techniques, including but not limited to filtration, distillation, crystallization, chromatography (i.e., HPLC) and the like. Such materials can be characterized using conventional means, including physical constants and spectral data (i.e., NMR, LCMS).

### EXAMPLES

### General Methods

Solvents, reagents, and intermediates that are commercially available were used as received. Reagents and intermediates that are not commercially available were prepared in the manner as described below. ¹H NMR spectra were obtained on a Bruker Avance 500 (500 MHz) and are reported as ppm downfield from Me₄Si with number of protons, multiplicities, and coupling constants in Hertz indicated parenthetically. Where LC/MS data are presented, analyses was performed using an Waters Acquity SQD LCMS instrument with an ACQUITY UPLC BEH C18 Column, 130 Å, 1.7 μm, 1 mm×50 mm, and a; gradient flow of mobile phase A (0.1% formic acid in water) and mobile phase B (0.1% formic acid in MeCN) at 0.300 mL/min at 50° C. Gradient: 90% A to 1% A from 0-1.6 minutes, hold 1% A from 1.6 to 3.0 minutes.

### Example 1

### Preparation of Intermediate Compound v

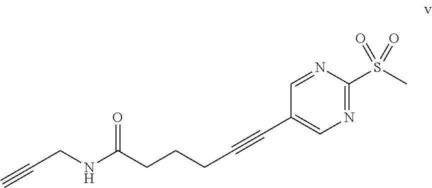 [SMILES: C#CCNC(=O)CCCC#Cc1cnc(S(C)(=O)=O)nc1]

Intermediate Compound v can be made, for example, using the methods described in US Patent Publication No. 2020/0347075.

### Example 2

### Alternate Preparation of Intermediate Compound v

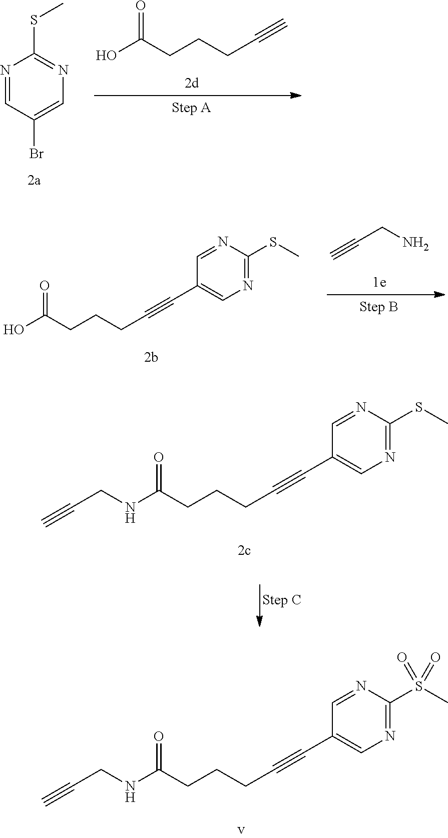

### Step A—Synthesis of Compound 2b

Compound 2a (100.0 g), compound 2d (60.0 g), and Cs₂CO₃ (794.0 g) were taken up in DMAc (1000 mL) at room temperature and put under a nitrogen atmosphere. To the resulting solution was added CuI (2.79 g), Pd₂(dba)₃ (0.45 g), dppf (0.54 g), and LiCl (20.67 g), and the resulting reaction was purged with nitrogen gas (3×), then heated to 55-65° C., and allowed to stir at this temperature for 20 hours. Water (1000 mL) was added to the reaction mixture, which was then stirred until the reaction mixture reached a temperature of 15-25° C. To this solution was added HCl (4M, 900 g) and water (2000 mL), and the resulting solution was cooled to 10-20° C., and allowed to stir at this temperature for 1 hour. The solution was filtered, and the filter cake was dried at 45-55° C. for 8 hours to provide compound 2b as a solid, which was used without further purification.

### Step B—Synthesis of Compound 2c

A solution of compound 2b (121.2 g) was taken up in DMAc (500 mL), and the resulting solution was allowed to stir at room temperature for 30 minutes. CDI (116.67 g) was added, and the resulting reaction was cooled to 0-10° C., and allowed to stir at this temperature for 16 hours. Prop-2-yn-amine (le, 35.0 g) was added, and the resulting reaction was allowed to stir at 0-10° C. for 1 hour. Water (1000 mL) was added, and the resulting solution was allowed to stir at 0-10° C. for 1 hour. Additional water was added (500 mL), and the resulting solution was allowed to stir at 0-10° C. for 1 hour. The solution was then filtered, and the filter cake was dried at 45-55° C. for 40 hours to provide compound 2c as a solid.

### Purification of Compound 2c

Compound 2c (100.10 g) was taken up in DCM (1500 mL), and to the resulting solution was added Ecosorb® C941. The resulting solution was cooled to 0-10° C., allowed to stir at this temperature for 2 hours, then filtered. The wet filter cake was washed with DCM (200 mL×3). The filtrate was concentrated to a volume of 300-400 mL in vacuo at 25-35° C., and n-heptane (1500 ml) was added. The resulting solution was cooled to 0-10° C., allowed to stir at this temperature for 2 hours, then filtered. The wet filter cake was washed with n-heptane (200 mL×2), and dried at 45-55° C. for 32 hours to provide compound 2c as solid.

### Step C—Synthesis of Compound v

To a solution of MeCN (800 mL) and water (800 mL) at 5-15° C., were added compound 2c (86.86 g) and oxone (402.66 g). The resulting reaction was allowed to stir at 5-15° C. for 16 hours, then ascorbic acid (1040 g, of a 20% aqueous solution, precooled to 0-10° C.) was added, and the resulting reaction was allowed to stir at 0-10° C. for 1 hour. The reaction mixture was filtered, and the wet filter cake was washed with DCM (500 mL), and to the combined filtrate and washings were added water (1600 mL) and DCM (400 mL). The organic layer was diluted with DCM (400 mL), and washed with water (2×800 mL), then the aqueous layer was extracted with DCM (400 mL). The organic layer and organic extracts were combined to provide compound Int-1 and the resulting solution was cooled to 20-30° C., and EcosorbC941 (8 g) was added. The resulting solution was allowed to stir for 30 minutes, then additional Ecosorb941 (8 g) was added, and the solution was allowed to stir for 1 hour. The solution was filtered, and the wet filter cake was washed with DCM (160 mL×2). The filtrate and washings were combined and concentrated in vacuo to a volume of 240 mL. A solution of n-heptane (400 mL) and DCM (80 mL) was added, and the resulting solution was agitated for 30 minutes. The resulting solution was filtered, and the wet filter cake was washed with n-heptane (2×160 mL), then dried in vacuo to provide compound v. ¹H NMR (500 MHz, DMSO) δ 9.12 (s, 2H), 8.31 (t, J=5.2 Hz, 1H), 3.86 (dd, J=5.5, 2.5 Hz, 2H), 3.41 (s, 3H), 3.08 (t, J=2.5 Hz, 1H), 2.56 (t, J=7.1 Hz, 2H), 2.28 (t, J=7.4 Hz, 2H), 1.82 (p, J=7.2 Hz, 2H).

### Example 3

### Preparation of Intermediate Compound xv

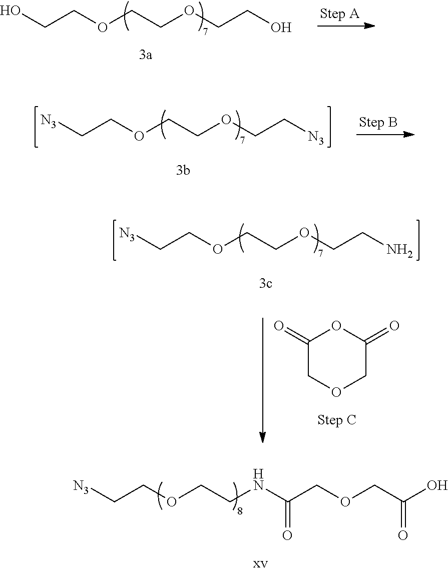

### Step A—Preparation of Compound 3b

Compound 3a (100.0 g) was taken up in toluene (346 g), and to the resulting solution was added DBU (95.5 g). The resulting mixture was allowed to stir for 1 hour at room temperature. In a separate vessel, DPPA (145.5 g) was taken up in toluene (260 g), and the resulting mixture was allowed to stir for 1 hour at room temperature. The DPPA solution was then added to the solution of compound A and heated at 100° C. The reaction was then mixed with a solution of NaCl (201 g) in water (1800 g), and the resulting mixture was allowed to stir for 1 hour at room temperature. The aqueous mixture was extracted with toluene (3×500 mL). The combined organic extracts were washed with 10% aqueous NaCl (2×500 mL), then concentrated in vacuo to approximately 600 mL. This solution, containing compound 3b, was used directly in the next step.

### Step B—Preparation of Compound 3c

To the solution of Compound 3b (573 g, prepared in Step A), was added additional toluene (2.3 L), and then a solution of sulfuric acid (46.4 g) in water (232 mL) was added. To the resulting solution was added triphenylphosphine (67.7 g) over a 2-hour period, while keeping the reaction temperature below 30° C. The reaction was then vented to release gas, then allowed to stir at room temperature for 16 hours. The reaction mixture as partitioned, and the aqueous phase was washed with DCM (2×1.1 L). To the combined organic phase and organic extracts was added 35% aqueous K₃PO₄ (954 g) until the solution was at pH 12-13. To this solution was added KH₂PO₄ (90 g) and THF (5.7 L) and the resulting solution was partitioned. The organic phase was collected and concentrated in vacuo to approximately 2 L, then THF (2.9 L) was added and the resulting solution was concentrated in vacuo to approximately 2 L, this THF addition and concentration step was repeated two more times, then to the final solution was added additional THF (2.9 L) to provide compound 3c in solution, which was used in the next step.

### Step C—Preparation of Compound xv

To a solution of Compound 4c (291.9 g, prepared in Step B) at room temperature was slowly added diglycolic anhydride (15.88 g, exothermic), and the resulting reaction was allowed to stir at room temperature for 10 minutes. To the reaction mixture was added DCM (1.5 L), and water (1.5 L). To the resulting solution was added 10% aqueous K₃PO₄ until the solution was at pH 7-8. The resulting solution was partitioned, the organic phase was discarded, and the aqueous phase was diluted with DCM (1.5 L). To the resulting solution was added IN H₂SO₄ to adjust the solution to pH 2-3. The resulting solution was partitioned, and the organic phase was concentrated in vacuo to approximately 600 mL to provide Intermediate Compound xv as a concentrated oil, which was used in the next step. ¹H NMR (400 MHz, CD3CN) δ 11.1 (broad, 1H), 7.14 (s, 1H), 4.18 (s, 2H), 4.09 (s, 2H), 3.68-3.49 (m, 32H), 3.40 (p, J=5.1 Hz, 4H).

Compound xv is also commercially available (CAS 846549-37-9).

### Example 4

### Preparation of Intermediate Compound iii

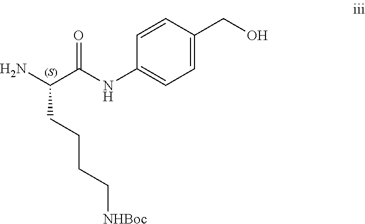

Intermediate Compound iii can be made, for example, using the methods described in US Patent Publication No. 2020/0347075.

### Example 5

### Preparation of Payload Compound xix

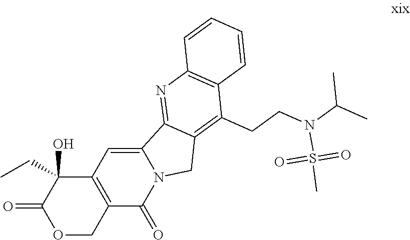 [SMILES: CC[C@@]1(O)C(=O)OCc2c1cc1n(c2=O)Cc2c-1nc1ccccc1c2CCN(C(C)C)S(C)(=O)=O]

Payload compound xix can be made, for example, using the methods described in US Patent Publication No. 2020/0347075.

### Example 6

### Alternate Preparation of Payload Compound xix

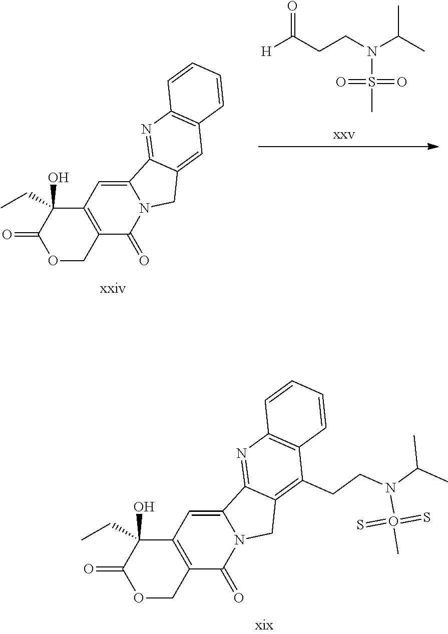

A vial containing xxiv ((S)-Camptothecin, 87 mg, 0.25 mmol) was charged with water (0.75 mL) and concentrated sulfuric acid (0.95 mL), and the resulting solution was cooled to 0° C. To this was added 0.25 mL of FeSO₄·7H₂O solution (prepared by taking up 120 mg of FeSO₄·7H₂O in 1 mL water), followed by an aqueous solution of xxv (0.75 mmol, 3 eq) in water (1 mL). To the resulting mixture was added hydrogen peroxide (30 wt %, 100 μL, 4 eq) dropwise, and the resulting reaction was allowed to stir at 0° C. for 1 hour. The reaction mixture was then warmed to room temperature, and dichloromethane (25 volumes) was added. To the resulting mixture was added water (12 volumes) dropwise, then the resulting solution was seeded with purified compound xix, and the mixture was allowed to stir for 4 hours at room temperature. To the resulting solution was added water (10 volumes) dropwise, and the resulting mixture was allowed to stir for 8 hours at room temperature. The resulting solution was filtered, and the collected solid was washed sequentially with water (2×2 volumes), DCM (2×2 volumes), and EtOH (2×2 volumes), then oven-dried at 50° C. to provide Compound xix. 1H NMR (400 MHz, DMSO) § 8.32 (d, J=8.0 Hz, 1H), 8.19 (dd, J=8.5, 1.0 Hz, 1H), 7.94-7.84 (m, 1H), 7.78 (ddd, J=8.2, 6.9, 1.3 Hz, 1H), 7.35 (s, 1H), 6.52 (s, 1H), 5.43 (m, 4H), 3.98 (m, 1H), 3.56-3.34 (m, 4H), 2.99 (s, 3H), 1.88 (m, 2H), 1.18-1.11 (m, 6H), 0.89 (t, J=7.3 Hz, 3H).

### Example 7

### Preparation of Linker Compound 1

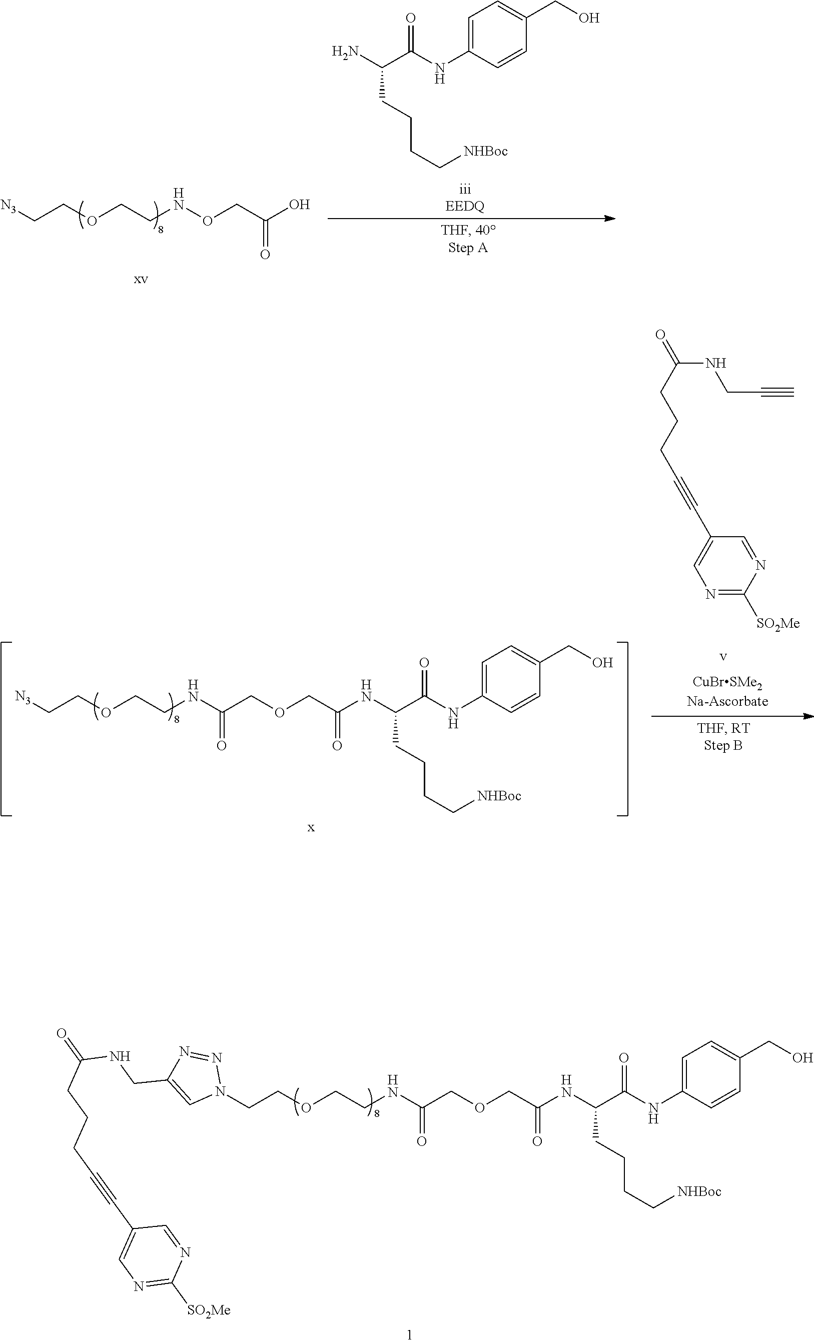

### Step A Synthesis of Compound x

To a room temperature solution of xv (10.6 g, 19.2 mmol, 1.00 equiv.) in THF (53 mL) under nitrogen atmosphere, was added iii (8.02 g, 23.0 mmol, 1.2 equiv.), followed by EEDQ (5.22 g, 21.1 mmol, 1.10 equiv.). The resulting reaction was heated to 40° C., and allowed to stir at this temperature for 16 hours. The reaction mixture was then filtered, and the collected solid was washed with THF (17 mL). The combined filtrate and washing was diluted with additional THF (15 mL) to provide a solution containing x, which was used directly in the next step.

### Step B—Synthesis of Linker Compound 1

To the solution of x obtained in Step A, at room temperature, was added v (6.45 g, 21.1 mmol, 1.10 equiv.), and the reaction vessel was purged with nitrogen for 5 minutes. CuBr—SMe₂ (197 mg, 0.959 mmol, 0.05 equiv.), and sodium ascorbate (190 mg, 0.959 mmol, 0.05 equiv.) were then sequentially added, and the resulting reaction was allowed to stir at room temperature for 16 hours. The reaction mixture was then diluted with THF (16 mL) and MeOH (342 mL) to reach a 3:1 MeOH/THF ratio, and Cuno-5 (11.4 g, 50 wt % with respect to Compound v) was added. The resulting reaction was allowed to stir at room temperature for 3 hours, then the reaction mixture was filtered through a pad of CELITE and the filtrate was collected to provide a solution of Compound 1 in THF (<10 ppm Cu). 180 mL of the filtrate (40% of the total material) was then purified using silica gel chromatography (0% to 15% MeOH/DCM) to provide Linker Compound 1 as an oil. ¹H NMR (400 MHz, CD₃CN) δ 8.90 (s, 2H), 8.80 (s, 1H), 7.72 (s, 1H), 7.52 (dd, J=13.4, 8.3 Hz, 3H), 7.27 (d, J=8.6 Hz, 2H), 7.17 (s, 1H), 7.00 (s, 1H), 5.39 (s, 1H), 4.51 (d, J=3.2 Hz, 2H), 4.49-4.41 (m, 3H), 4.39 (d, J=5.7 Hz, 2H), 4.07-3.99 (m, 4H), 3.86-3.76 (m, 2H), 3.58-3.47 (m, 30H), 3.37 (q, J=5.6 Hz, 2H), 3.29 (s, 4H), 3.00 (q, J=6.5 Hz, 2H), 2.54 (t, J=7.1 Hz, 2H), 2.33 (t, J=7.4 Hz, 2H), 1.90 (q, J=7.2 Hz, 3H), 1.74 (dq, J=9.4, 4.1 Hz, 1H), 1.38 (s, 14H). MS: Calculated: [M+H]⁺ 1193.6, Observed: 1193.7

### Step C (Optional)—Adsorption of Linker Compound 1 on Solid Phase Support

To a solution of Linker Compound 1 in MeCN (2.5V relative to the solution of Compound 1) is added IPAc (3× volume of MeCN). The resulting suspension is stirred at room temperature, and Celite-545 (300 wt % relative to Compound 1) is added, then additional IPAC (2.5V relative to Compound 1) is added dropwise over 15 minutes. Stirring is stopped, and the resulting slurry is allowed to age at room temperature for 30 minutes, then filtered. The collected solid was washed with 2-MeTHF (3×4V relative to Compound 1), then dried under vacuum under nitrogen atmosphere to provide Linker Compound 1 adsorbed onto Celite.

### Example 8

### Preparation of Linker-Payload A

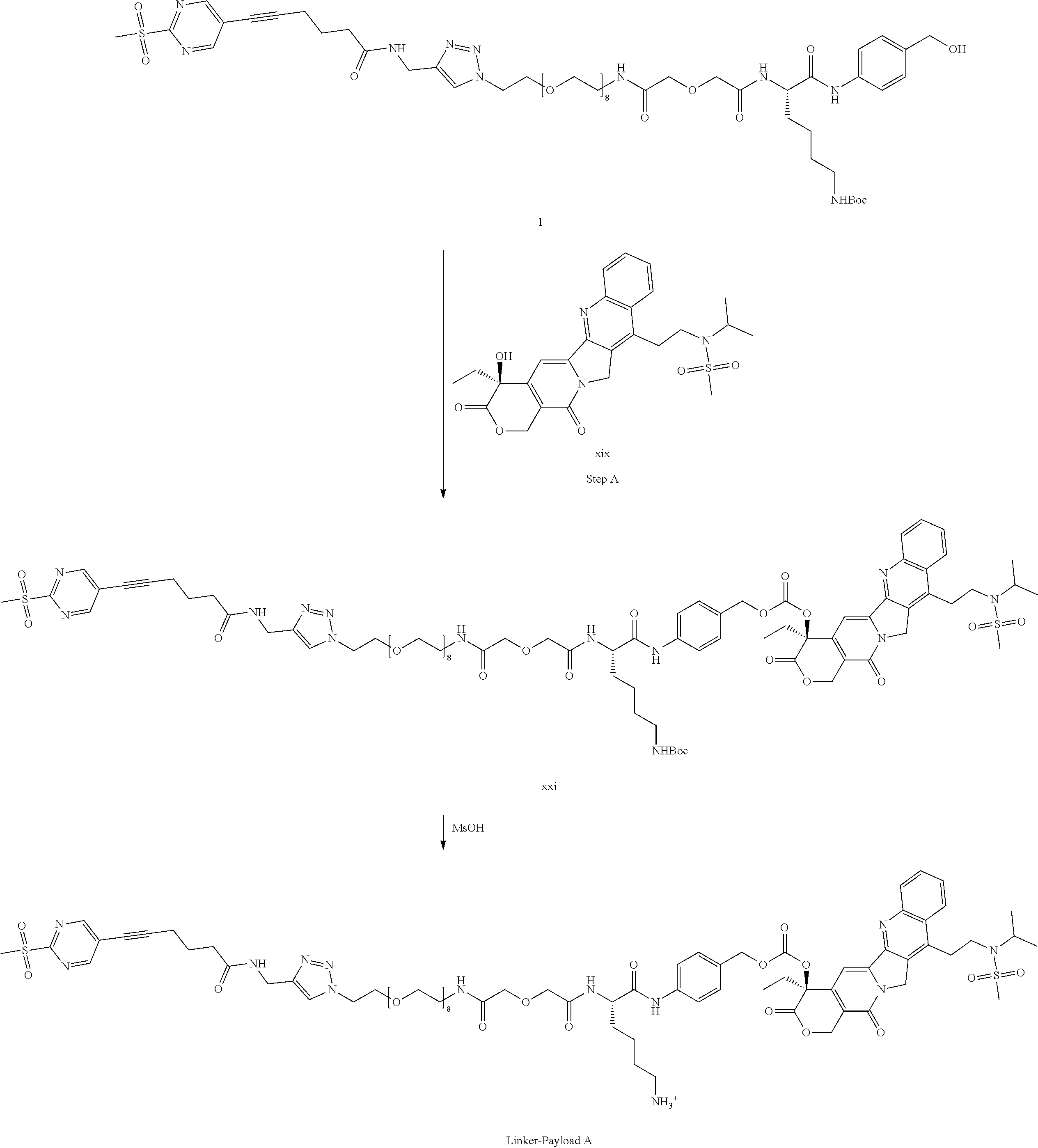

### Step A—Synthesis of Compound xxi

To a 40 mL vial was added payload Compound xix (485 mg, 0.948 mmol) and 5 mL MeCN. The resulting solution was cooled to −20° C., and triphosgene (93 mg, 0.313 mmol) was added, followed by dropwise addition of pyridine (192 μl, 2.370 mmol). The mixture was allowed to stir at −20° C. for 45 minutes, then a solution of linker Compound 1 (1301 mg, 1.090 mmol) in 5 mL MeCN was added dropwise. The resulting reaction was allowed to stir at −20° C. for 30 minutes, the cold bath was removed, and the reaction mixture was allowed to warm to room temperature. then stirred overnight. The resulting reaction mixture was then filtered, and the filtrate was concentrated in vacuo. The residue obtained was purified using column chromatography (5% MeOH in DCM eluent), to provide Compound xxi as a solid. Calculated exact mass: 1729.73. Observed mass: 1730.73 (M+H).

### Step B—Deprotection of xxi

A solution of compound xxi was prepared in MeCN solution (100 mg/mL concentration). To 3 mL of this solution was added methanesulfonic acid (100 mg, 67.3 μL), and the resulting reaction was allowed to age at 25° C. for 2 hours. 5-Ethyl-2-Methylpyridine (167 mg, 182 μL) was then added, and the resulting mixture was cooled to room temperature. The reaction mixture was directly purified using HPLC (Kromasil 60-10 diol resin and eluting with a mixture of MeCN, tBuOH, and MeOH). An HPLC fraction containing 216 mg of compound A in a mixture of acetonitrile: tert-butanol: methanol was concentrated and solvent switched into 1,3 dioxolane to 94 mg/mL concentration. The resulting solution was added dropwise to tert-butyl acetate (21 mL) to precipitate the product, and the resulting slurry was allowed to stir at room temperature for 3 hours. The slurry was then filtered, and the collected solid was dried over a stream of nitrogen to provide Linker-Payload A as a solid. Product identify was confirmed by HPLC using an authentic product standard. Observed mass: 1631.1 (M+H).

### Example 9

### Alternate Preparation of Linker-Payload A on Celite Support

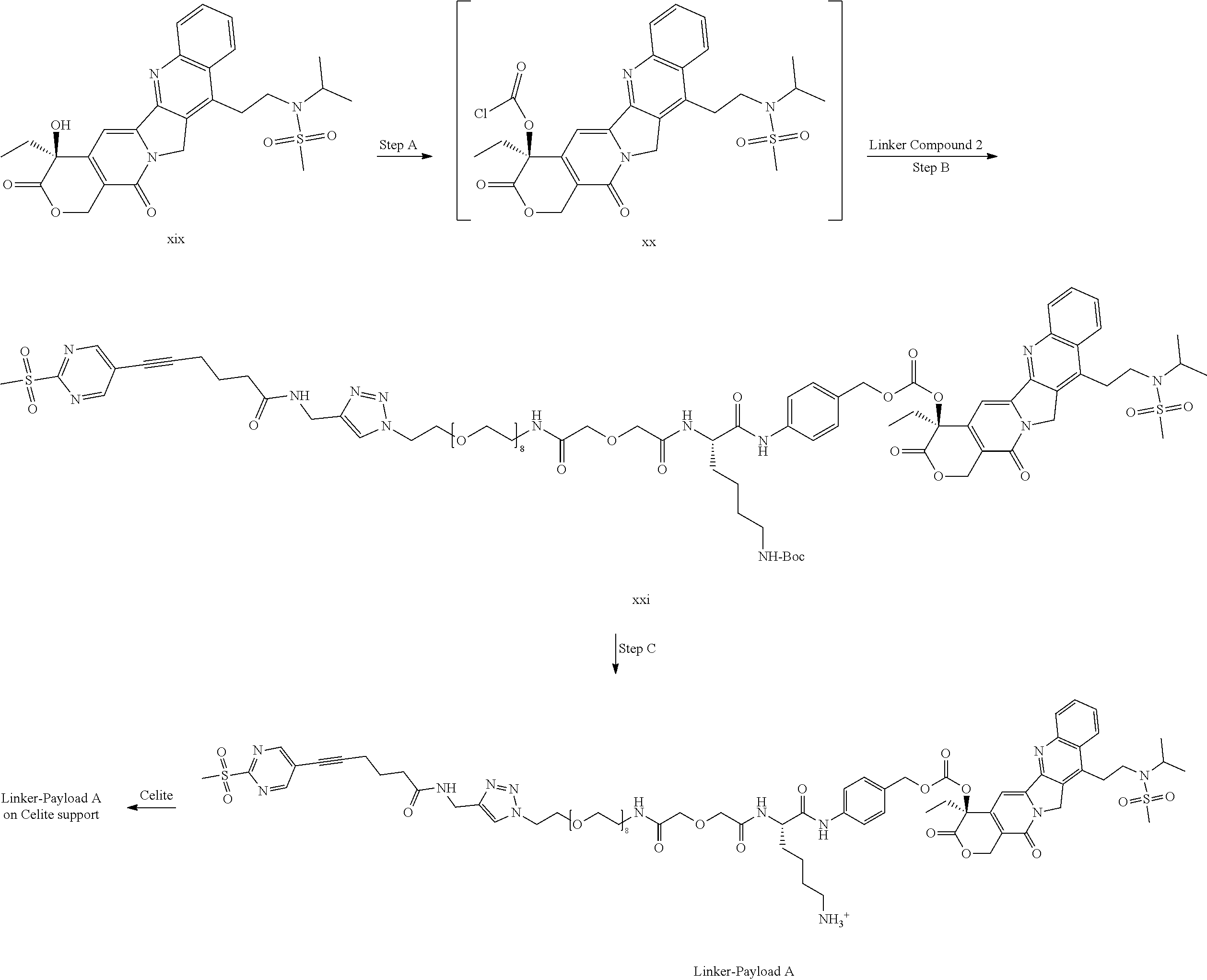

### Step A—Synthesis of Payload Intermediate xx

Payload compound xix (21.4 g) was slurried in MeCN (298 mL), and to the resulting slurry was added pyridine (7.6 mL), then the resulting mixture was cooled to −10° C. Triphosgene (4.1 g) was added and the resulting reaction was allowed to age at −10° C. for 4 hours. The resulting solution, containing compound xx, was used directly in the next step.

### Step B—Synthesis of Payload/Linker Intermediate xxi

A solution of linker compound 1 (33.1 g) in MeCN (139 mg/mL, KF<200 ppm) was added to the solution of payload intermediate xx (prepared in Step A), the resulting reaction was allowed to age at −10° C. for approximately 18 hours, and the reaction was quenched with water (2.5 mL). The resulting mixture was warmed to 20° C., filtered, rinsed with MeCN (33 mL) and EtOAc (33 mL). The filtrate was diluted with EtOAc (264 mL), and washed sequentially with 5% aqueous NaMSA (2×265 mL) and water (132 mL). The EtOAc phase (containing linker/payload intermediate compound xxi) was collected, and solvent switched through distillation to MeCN to a final volume of 297 mL.

### Step C—Synthesis of Payload/Linker Compound xxi on Celite

The MeCN solution containing compound 8a (prepared in Step B), was diluted with MTBE (132 mL), and to the resulting solution was added celite (165 g). Additional MTBE (462 mL) was slowly added, and the resulting slurry was filtered. The collected solid was washed with a mixture of MTBE: MeCN (248 mL MTBE, 99 mL MeCN), and dried in vacuo to provide drug/linker compound xxi adsorbed onto Celite support, 203 g, 18.6 wt %.

While the foregoing specification teaches the principles of the present disclosure, with examples provided for the purpose of illustration, the practice of the processes of the present disclosure encompasses all of the usual variations, adaptations and/or modifications that come within the scope of the following claims. All publications, patents and patent applications cited herein are incorporated by reference in their entirety into the disclosure.

What is claimed is:

1. A process for making a compound of structural formula (I): 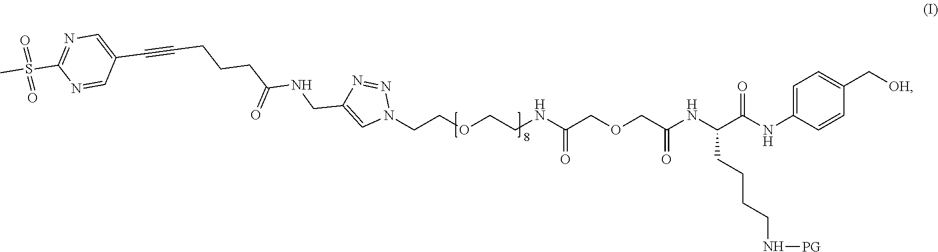
wherein PG is a primary amine protecting group,
comprising the steps:
(A) contacting a compound of structural formula (i): 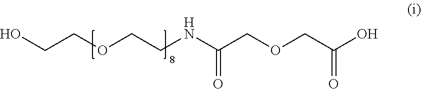
with a deoxychlorinating agent, in organic solvent A, at a temperature and for a time sufficient to form a compound of structural formula (ii): 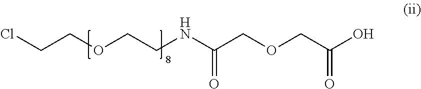
wherein organic solvent A is selected from dichloromethane, toluene, THF, acetonitrile, and mixtures thereof;
(B) contacting the product of Step A with a compound of structural formula (iii): 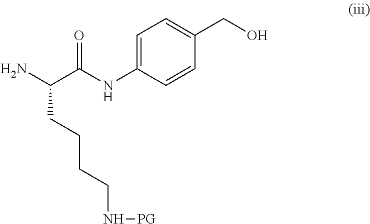
with an amide coupling agent, in the presence of a base in organic solvent B, at a temperature, and for a time sufficient to form a compound of structural formula (iv): 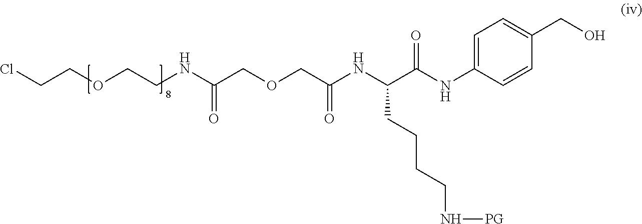
organic solvent B is selected from methyl ethyl ketone, acetone, dichloroethane, dimethyl ether, diethyl ether, methyl isobutyl ketone, toluene, THF, DCM, MTBE, DMF, propylene carbonate, DME, 1,3-dimethyl-3,4,5,6-tetrahydro-2(1H)-pyrimidinone, 2-methyltetrahydrofuran, xylenes, ethyl acetate, NMP, anisole, isopropyl acetate, acetonitrile and mixtures thereof; and
(C) contacting the product of step B with a compound of structural formula (v): 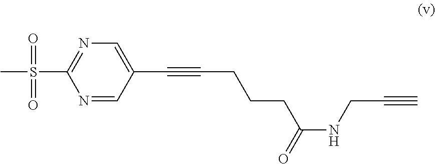 [SMILES: C#CCNC(=O)CCCC#Cc1cnc(S(C)(=O)=O)nc1]
in the presence of a copper salt, and an azide anion source, in organic solvent C, at a temperature and for a time sufficient to form the compound of structural formula (I), wherein organic solvent C is selected from THF, acetonitrile, toluene, methyl THF, isopropyl acetate, dichloromethane, water, and mixtures thereof.

2. The process of claim 1, wherein the deoxychlorinating agent used in Step (A) is selected from SOCl₂, PCl₃, PCl₅, POCl₃, oxalyl chloride, tosyl chloride, mesyl chloride, triphenylphosphine/CCl₄, and triphenyl phosphine/hexachloroethane;
the amide coupling agent used in Step (B) is selected from HATU, EDC, DCC, HBTU, BOP, COMU, and TSTU;
the base used in Step (B) is selected from a carbonate base, TEA, DIPEA, DBU, pyridine, and 2-picoline;
the copper salt used in Step (C) is selected from CuCl, CuBr, CuBr—SMe₂, CuI, Cu(OAc), Cu(OAc)₂, CuSO₄, Cu₃(PO₄)₂;
the azide anion source in Step (C) is NaN₃ or KN₃; and
the primary amine protecting group is selected from Boc, MMT, Cbz, and Fmoc.

3. A process for making a compound of structural formula (I): 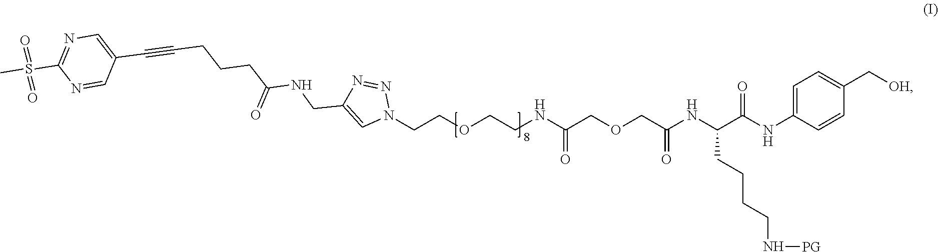
wherein PG is a primary amine protecting group,
comprising the steps:
(A) contacting a compound of structural formula (vi): 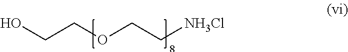
with a deoxychlorinating agent, in organic solvent D, at a temperature and for a time sufficient to form a compound of structural formula (vii): 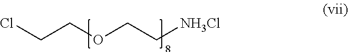
wherein organic solvent D is selected from dichloromethane, toluene, THF, acetonitrile, and mixtures thereof;
(B) contacting the product of Step A with diglycolic anhydride (viii): 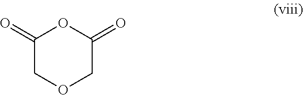
in organic solvent E, at a temperature, and for a time sufficient to form a compound of structural formula (ix): 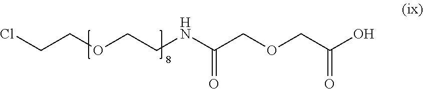
wherein organic solvent E is selected from THF, acetonitrile, toluene, methyl THF, isopropyl acetate, ethyl acetate, dichloromethane, and mixtures thereof;
(C) contacting the product of Step B with a compound of structural formula (iii): 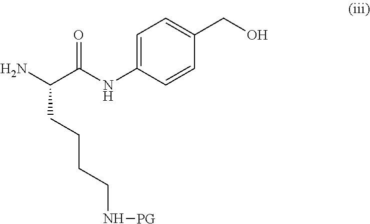
with an amide coupling agent, in the presence of a base in organic solvent F, at a temperature, and for a time sufficient to form a compound of structural formula (iv): 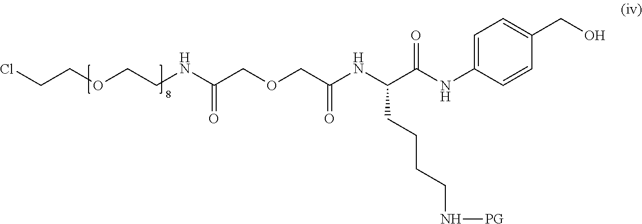
wherein organic solvent F is selected from methyl ethyl ketone, acetone, dichloroethane, dimethyl ether, diethyl ether, methyl isobutyl ketone, toluene, THF, DCM, MTBE, DMF, propylene carbonate, DME, 1,3-dimethyl-3,4,5,6-tetrahydro-2(1H)-pyrimidinone, 2-methyltetrahydrofuran, xylenes, ethyl acetate, NMP, anisole, isopropyl acetate, acetonitrile and mixtures thereof; and
(D) contacting the product of Step C with an azide anion, in organic solvent G, at a temperature and for a time sufficient to form an intermediate compound of structural formula (x): 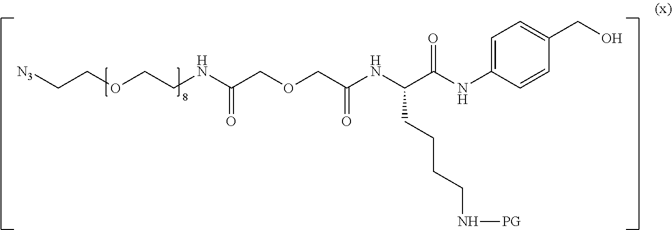
then contacting intermediate compound (x) with a compound of structural formula (v): 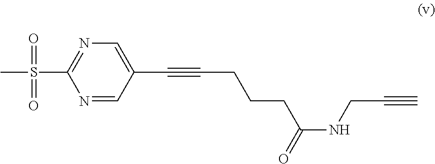 [SMILES: C#CCNC(=O)CCCC#Cc1cnc(S(C)(=O)=O)nc1]
in the presence of a copper salt, and an azide anion source, at a temperature and for a time sufficient to form the compound of structural formula (I), wherein organic solvent G is selected from THF, acetonitrile, toluene, methyl THF, isopropyl acetate, ethyl acetate, dichloromethane, water, and mixtures thereof.

4. The process of claim 3, wherein:
the deoxychlorinating agent used in Step (A) is selected from SOCl₂, PCl₃, PCl₅, POCl₃, oxalyl chloride, tosyl chloride, mesyl chloride, triphenylphosphine/CCl₄, and triphenyl phosphine/hexachloroethane;
the amide coupling agent used in Step (C) is selected from HATU, EDC, DCC, HBTU, BOP, COMU, and TSTU;
the base used in Step (C) is selected from a carbonate base, TEA, DIPEA, DBU, pyridine, and 2-picoline;
the azide anion source in Step (D) is NaN₃ or KN₃;
the copper salt used in Step (D) is selected from CuCl, CuBr, CuBr—SMe₂, CuI, Cu(OAc), Cu(OAc)₂, CuSO₄, Cu₃(PO₄)₂; and
the primary amine protecting group is selected from Boc, MMT, Cbz, and Fmoc.

5. A process for making a compound of formula (I): 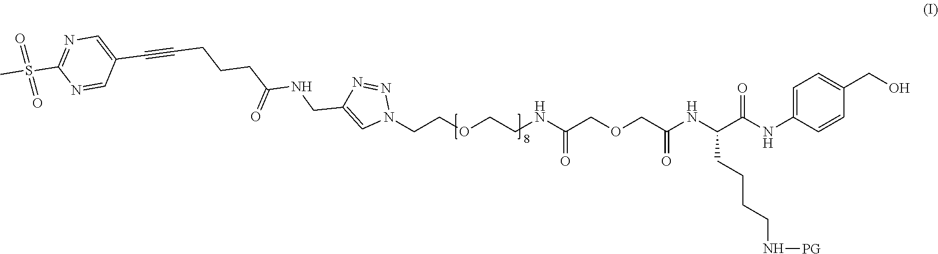
wherein PG is a primary amine protecting group,
comprising the steps:
(A) contacting a compound of structural formula (ii): 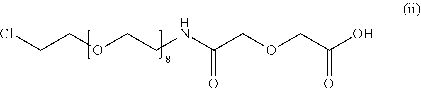
with an azide anion in organic solvent J, at a temperature and for a time sufficient to form an intermediate compound of structural formula (xv): 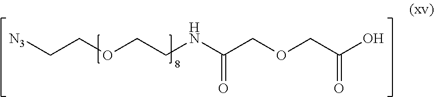
then, contacting the intermediate compound (xv) with a compound of structural formula (v): 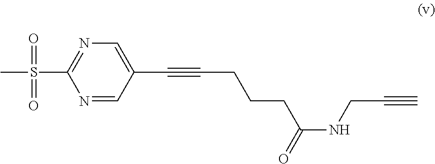 [SMILES: C#CCNC(=O)CCCC#Cc1cnc(S(C)(=O)=O)nc1]
in the presence of a copper salt, at a temperature and for a time sufficient to form a compound of structural formula (xvi): 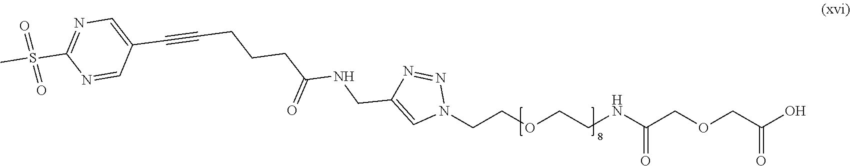
wherein organic solvent J is selected from THE, acetonitrile, toluene, methyl THF, isopropyl acetate, ethyl acetate, dichloromethane, water, and mixtures thereof, and
(B) contacting the product of Step A with a compound of structural formula (iii): 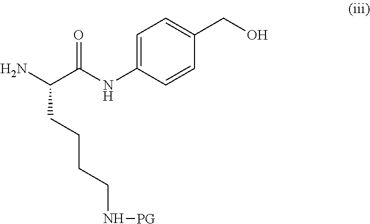
in the presence of an amide coupling agent and a base in organic solvent K, at a temperature, and for a time sufficient to form the compound of structural formula (I), wherein organic solvent K is selected from THE, acetonitrile, toluene, methyl THF, isopropyl acetate, ethyl acetate, dichloromethane, and mixtures thereof.

6. The process of claim 5, wherein:
the source of the azide anion in Step (A) is NaN₃ or KN₃;
the copper salt used in Step (A) is selected from CuCl, CuBr, CuBr—SMe₂, CuI, Cu(OAc), Cu(OAc)₂, CuSO₄, and Cu₃(PO₄)₂;
the amide coupling agent used in Step (B) is selected from HATU, EDC, DCC, HBTU, BOP, COMU, and TSTU;
the base used in Step (B) is selected from a carbonate base, TEA, DIPEA, DBU, pyridine, and 2-picoline; and
the primary amine protecting group is selected from Boc, MMT, Cbz, and Fmoc.

7. A process for making a compound of formula (I): 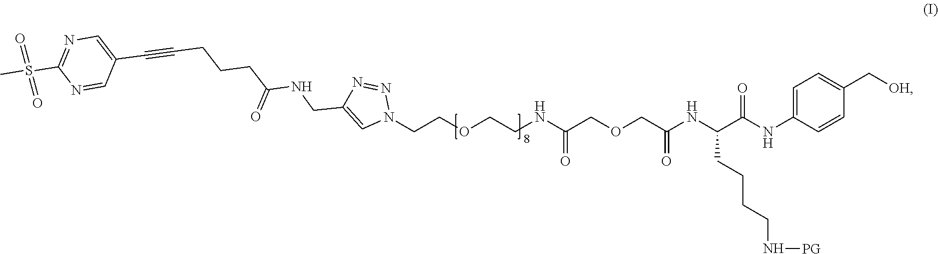
wherein PG is a primary amine protecting group,
comprising the steps:
(A) contacting a compound of structural formula (xi): 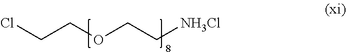
with an azide anion, in organic solvent H, at a temperature and for a time sufficient to form an intermediate compound of structural formula (xii): 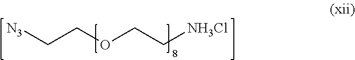
then, contacting the intermediate compound (xii) with a compound of structural formula (v): 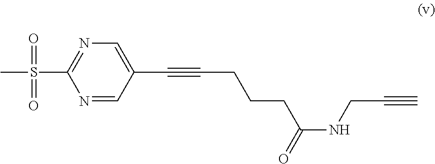 [SMILES: C#CCNC(=O)CCCC#Cc1cnc(S(C)(=O)=O)nc1]
in the presence of a copper salt, at a temperature and for a time sufficient to form compound of structural formula (xiii): 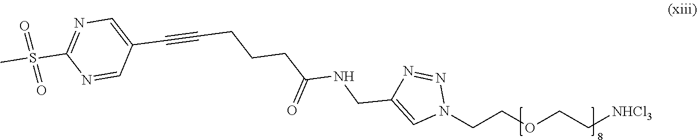
wherein organic solvent H is selected from THE, acetonitrile, DMF, toluene, methyl THF, isopropyl acetate, ethyl acetate, dichloromethane, water, and mixtures thereof, and
(B) contacting the product of Step A with a compound of structural formula (xiv): 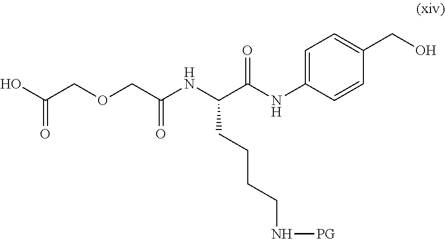
in the presence of an amide coupling agent, and a base in organic solvent I at a temperature, and for a time sufficient to form the compound of structural formula (I), wherein the organic solvent I is selected from THE, acetonitrile, toluene, methyl THF, isopropyl acetate, ethyl acetate, dichloromethane, and mixtures thereof.

8. The process of claim 7, wherein:
the source of the azide anion in Step (A) is NaN₃ or KN₃;
the copper salt used in Step (A) is selected from CuCl, CuBr, CuBr—SMe₂, CuI, Cu(OAc), Cu(OAc)₂, CuSO₄, Cu₃(PO₄)₂;
the amide coupling agent used in Step (B) is selected from HATU, EDC, DCC, HBTU, BOP, COMU, and TSTU;
the base used in Step (B) is selected from a carbonate base, TEA, DIPEA, DBU, pyridine, and 2-picoline; and
the primary amine protecting group is selected from Boc, MMT, Cbz, and Fmoc.

9. A process for making a compound of structural formula (I): 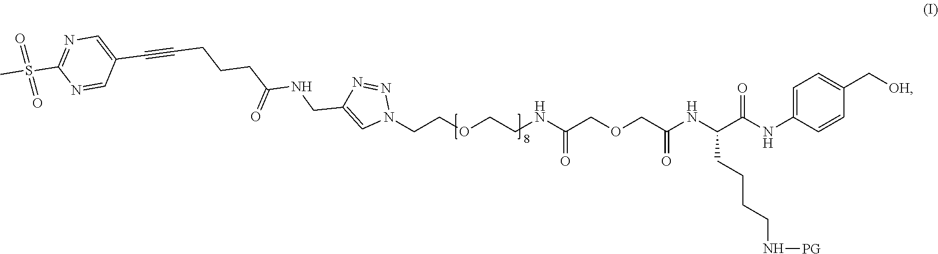
wherein PG is a primary amine protecting group,
comprising the steps:
(A) contacting a compound of structural formula xv: 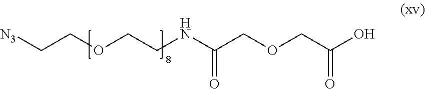
with a compound of structural formula iii: 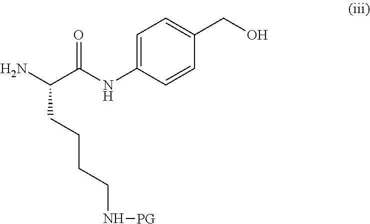
wherein PG is a primary amine protecting group, with an amide coupling agent, in organic solvent L, at a temperature, and for a time sufficient to form a compound of structural formula x: 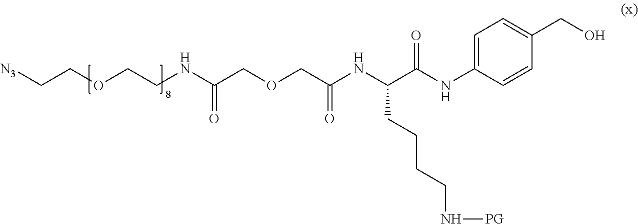
organic solvent A is selected from methyl ethyl ketone, acetone, dichloroethane, dimethyl ether, diethyl ether, methyl isobutyl ketone, toluene, THF, DCM, MTBE, DMF, propylene carbonate, DME, 1,3-dimethyl-3,4,5,6-tetrahydro-2(1H)-pyrimidinone, 2-methyltetrahydrofuran, xylenes, ethyl acetate, NMP, anisole, isopropyl acetate, acetonitrile and mixtures thereof; and
(B) contacting the product of step A with a compound of structural formula v: 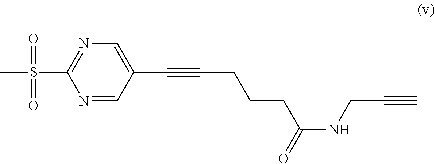 [SMILES: C#CCNC(=O)CCCC#Cc1cnc(S(C)(=O)=O)nc1]
in the presence of a copper salt in organic solvent M, at a temperature and for a time sufficient to form the compound of structural formula (I), wherein organic solvent M is selected from THF, acetonitrile, toluene, methyl THF, isopropyl acetate, dichloromethane, water, and mixtures thereof.

10. The process of claim 9, wherein the amide coupling agent used in Step (A) is selected from EEDQ, HATU, EDC, DCC, HBTU, BOP, COMU, and TSTU;
the copper salt used in Step (B) is selected from CuCl, CuBr, CuBr—SMe₂, CuI, Cu(OAc), Cu(OAc)₂, CuSO₄, Cu₃(PO₄)₂; and
the primary amine protecting group is selected from Boc, MMT, Cbz, and Fmoc.

11. A process for making a compound of structural formula xix: 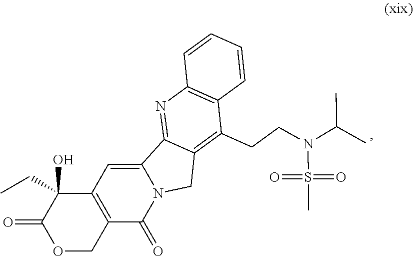 [SMILES: CC[C@@]1(O)C(=O)OCc2c1cc1n(c2=O)Cc2c-1nc1ccccc1c2CCN(C(C)C)S(C)(=O)=O]
comprising contacting a compound of structural formula (xx): 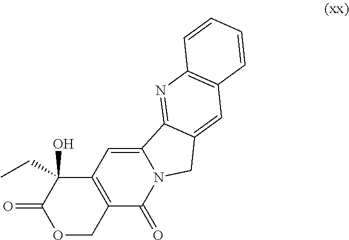 [SMILES: CC[C@@]1(O)C(=O)OCc2c1cc1n(c2=O)Cc2cc3ccccc3nc2-1]
with a compound of structural formula xxv: 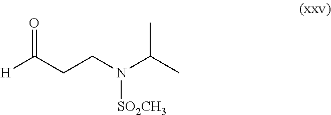
in the presence of an acid, a metal salt, and an oxidizing agent, in a solvent P, at a temperature, and for a time sufficient to form the compound of structural formula (xix), wherein solvent P is selected from water, MeCN, acetic acid, trifluoroethanol, NMP, propylene glycol, trifluoroacetic acid, and mixtures thereof.

12. The process of claim 11, wherein:
the acid used is selected from H₂SO₄, TFA, phosphoric acid, sulfonic acid, and methanesulfonic acid;
the metal salt used is selected from Fe₂SO₄, V(acac)₃, V₂O₃, NaVO₃, FeCl₃, and FeO; and
the oxidizing agent used is selected from H₂O₂, a persulfate, peracetic acid, cumene hydrogen peroxide, 2-butanone peroxide, oxone, urea hydrogen peroxide, sodium perborate, di-t-butyl peroxide, dibenzoyl peroxide, and sodium hypochlorite.

13. A process for making Linker-Payload A: 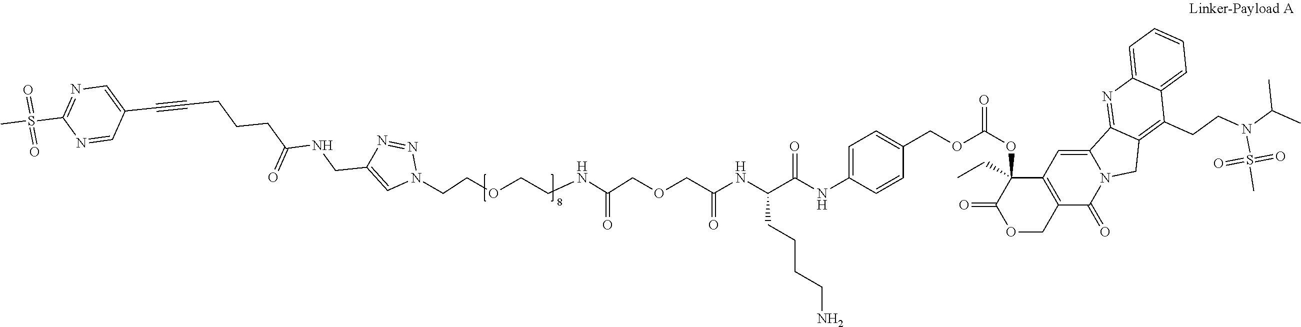
comprising the steps:
(A) contacting a compound of structural formula xix: 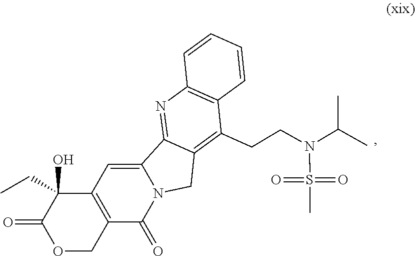 [SMILES: CC[C@@]1(O)C(=O)OCc2c1cc1n(c2=O)Cc2c-1nc1ccccc1c2CCN(C(C)C)S(C)(=O)=O]
with phosgene or triphosgene, in the presence of a base, in organic solvent N, at a temperature and for a time sufficient to form a compound of structural formula xx: 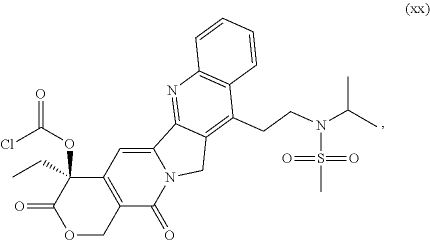 [SMILES: CC[C@@]1(OC(=O)Cl)C(=O)OCc2c1cc1n(c2=O)Cc2c-1nc1ccccc1c2CCN(C(C)C)S(C)(=O)=O]
wherein organic solvent N is selected from methyl ethyl ketone, acetone, dichloroethane, dimethyl ether, diethyl ether, methyl isobutyl ketone, toluene, THF, DCM, MTBE, propylene carbonate, DME, 1,3-dimethyl-3,4,5,6-tetrahydro-2(1H)-pyrimidinone, 2-methyltetrahydrofuran, xylenes, ethyl acetate, NMP, anisole, isopropyl acetate, acetonitrile and mixtures thereof; and
(B) contacting the product of Step A with a compound of structural formula (I): 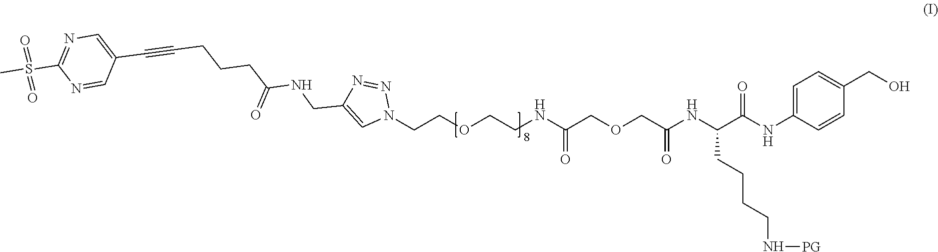
in organic solvent O, at a temperature and for a time sufficient to form a compound of structural formula xxi: 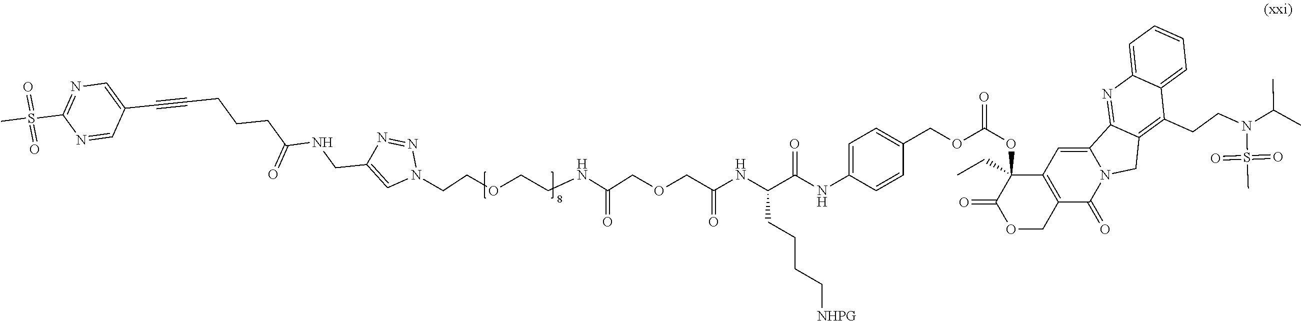
wherein organic solvent N is selected from methyl ethyl ketone, acetone, dichloroethane, dimethyl ether, diethyl ether, methyl isobutyl ketone, toluene, THF, DCM, MTBE, propylene carbonate, DME, 1,3-dimethyl-3,4,5,6-tetrahydro-2(1H)-pyrimidinone, 2-methyltetrahydrofuran, xylenes, ethyl acetate, NMP, anisole, isopropyl acetate, acetonitrile and mixtures thereof; and
(C) removing the protecting group from the product of Step B to provide Linker-Payload A:

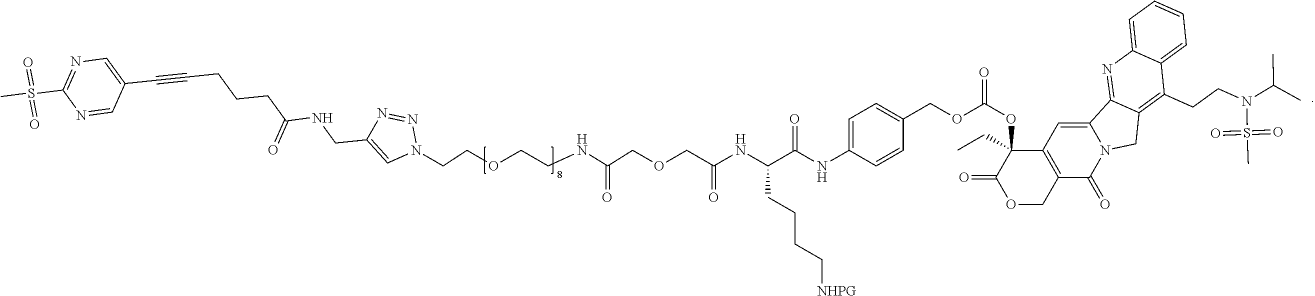

14. The process of claim 13, wherein:
the base used in Step A is selected from a carbonate base, TEA, DIPEA pyridine, and 2-picoline;
the primary amine protecting group is selected from Boc, MMT, Cbz, and Fmoc; and
the primary amine protecting group is removed in Step (C) using TFA or methanesulfonic acid.

15. The process of claim 1, wherein the compound of formula (I) is adsorbed onto a solid support.

16. The process of claim 13, wherein the compound of formula xxi is adsorbed onto a solid support.

17. The process of claim 13, wherein Linker-Payload A is adsorbed onto a solid support.

18. The process of claim 15, wherein the solid support is Celite.

19. A compound selected from: 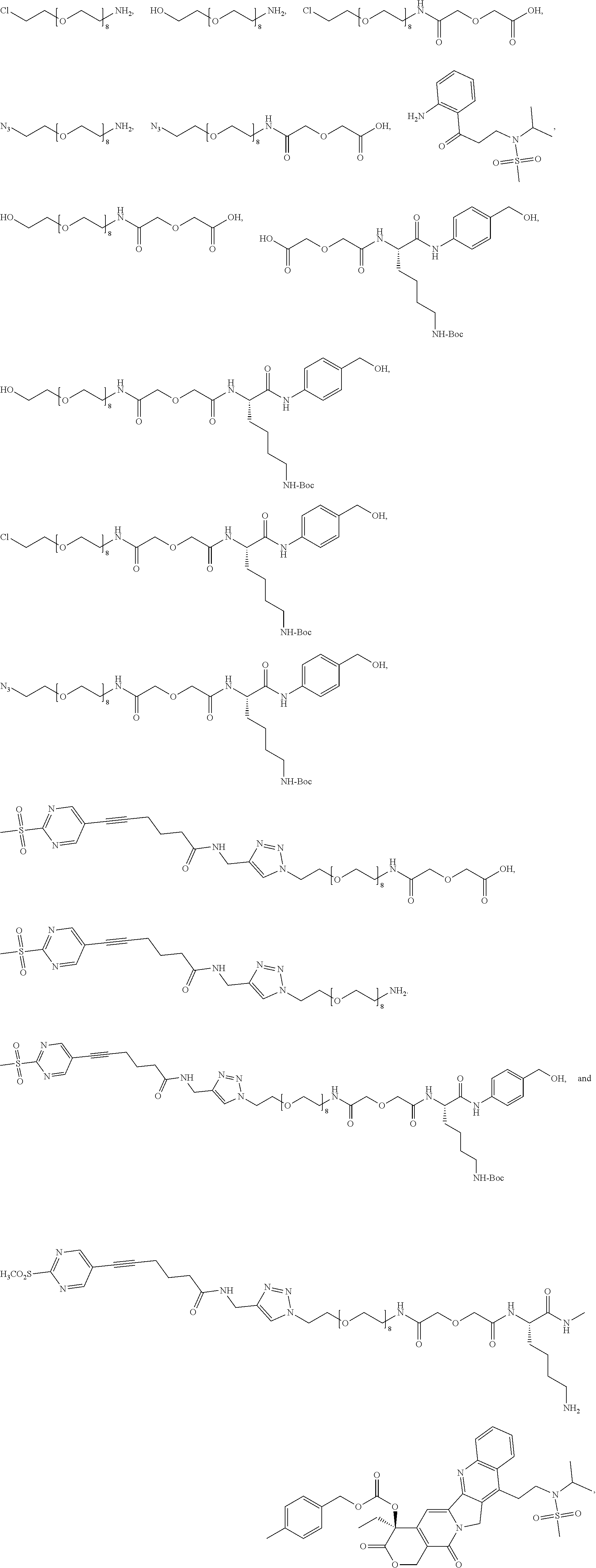
or a salt thereof.
